(19) United States
(12) Patent Application Publication
(10) Pub. No.: US 2026/0263354 A1
(43) Pub. Date: Sep. 10, 2026

(54) BIODISSOLVABLE MICRONEEDLE ARRAY-METAL-ORGANIC FRAMEWORK-VACCINE BIOCOMPOSITES FOR EFFECTIVE SKIN IMMUNIZATION AND MANUFACTURING THEREOF

(71) Applicant: University of Pittsburgh - Of the Commonwealth System of Higher Education, Pittsburgh, PA (US)

(72) Inventors: Louis D. Falo, Bradfordwoods, PA (US); Emrullah Korkmaz, Pittsburgh, PA (US); Nathaniel L. Rosi, Pittsburgh, PA (US); Zoe M. Soilis, Pittsburgh, PA (US); Stephen C. Balmert, Cranberry Township, PA (US)

(73) Assignee: University of Pittsburgh - Of the Commonwealth System of Higher Education, Pittsburgh, PA (US)

(21) Appl. No.: 19/165,553

(22) PCT Filed: Mar. 15, 2024
(86) PCT No.: PCT/US2024/020166

§ 371 Date: Sep. 15, 2025

Related U.S. Application Data
(60) Provisional application No. 63/490,670, filed on Mar. 16, 2023.

(51) Int. Cl. A61K 9/00; A61K 9/51; A61M 37/00; B82Y 5/00

(52) U.S. Cl. CPC A61K 9/0021; A61K 9/5115; A61K 9/5123; A61K 9/5161; A61M 37/0015; A61M 2037/0023; A61M 2037/0046; A61M 2037/0053; B82Y 5/00

(57) ABSTRACT

Disclosed herein are aspects of a microneedle array comprising a metal-organic framework (MOF)-vaccine biocomposite and methods for using the same. The microneedle array further comprises a dissolvable material that dissolved when inserted into the skin of a subject, thereby releasing the MOF-vaccine biocomposite. The MOF may be selected to dissolve in an acidic environment, thereby targeting the vaccine delivery to specific cellular compartments. Methods for making the MOF-vaccine biocomposite and the microneedle array also are disclosed.

### CROSS REFERENCE TO RELATED APPLICATION

This application claims the benefit of the earlier filing date of U.S. provisional patent application No. 63/490,670, filed Mar. 16, 2023, which is incorporated herein by reference in its entirety.

### ACKNOWLEDGMENT OF GOVERNMENT SUPPORT

This invention was made with government support under Grant No. R01 AR079233 awarded by the National Institutes of Health. The government has certain rights in the invention.

### FIELD

This disclosure is directed to microneedle arrays comprising a metal-organic framework for effective vaccination.

### BACKGROUND

Vaccines provide a simple, safe, and effective way of achieving protection against diseases, including preventing infections or preventing or reducing the severity of symptoms of infectious diseases. Vaccines are often administered by injection using hypodermic needles, which pose significant challenges associated with vaccine administration, immunogenicity, safety, and logistics for effective global immunization campaigns. Further, each vaccine composition requires a specific formulation suitable for the specific antigens contained therein. For example, solvents, buffers, stabilizers, and adjuvants need to be selected such that the antigen does not react or degrade in the formulation before administration and provides potent immunogenicity. During vaccine delivery, certain antigens may be adversely affected in the extracellular space before the vaccine composition is internalized into cells within the patient to produce a desired immunological effect and during vaccine storage vaccine components may be adversely affected due to environmental stressors. Thus, there is a need for alternative vaccine formulation and delivery technologies that can overcome these issues with vaccine formulation, delivery, storage, and distribution.

### SUMMARY

Disclosed herein is a composite microneedle array that addresses the issues associated with vaccine formulation, delivery, storage, and distribution. In some aspects, the microneedle array comprises a base portion and a plurality of biocompatible dissolvable microneedles extending from the base portion, wherein the microneedles comprise metal-organic framework (MOF) particles comprising a vaccine composition. The MOF may be a zinc-based MOF such as ZIF-8, ZIF-10, ZIF-90, a copper-based MOF such as HKUST-1, an iron-based MOF such as MIL-88, magnesium-based MOF, chromium-based MOF, calcium-based MOF, europium-based MOF, bismuth-based MOF, titanium-based MOF, cobalt-based MOF, nickel-based MOF, or zirconium-based MOF.

In any aspects, the MOF may be surface activated, such as with a surface coating comprising PEG, PVA, PVP, hyaluronic acid, silk, or a combination thereof. And/or the MOF particles may have an average size distribution of from 10 nm to 50,000 nm.

In some aspects, the base portion of the microneedle array lacks the MOF particles comprising the vaccine composition. In other examples, both the microneedles and the base portion include the MOF particles comprising the vaccine composition. And in certain aspects, the MOF particles comprising the vaccine composition are integrated into an apex portion of the microneedles.

In some aspects, the MOF particles comprising the vaccine composition are premade and then loaded into the production molds. However, in other aspects, the MOF particles comprising the vaccine composition are synthesized in the production molds.

In some aspects, the vaccine composition comprises an antigen selected from protein, mRNA, self-replicating mRNA, nucleic acid antigen, recombinant viral vectored antigen, nucleic acid origami antigen, extracellular vesicle antigen, inactivated virus antigen, liposome encapsulated antigen, extracellular vesicle encapsulated antigen, cell lysate antigen, bacterial antigen, or a combination thereof. Additionally, or alternatively, the vaccine composition comprises an adjuvant that can be a small-molecule immune potentiator, such as saponin; a STING pathway agonist; or a nucleic acid and nucleic acid origami immune potentiator, such as a single-stranded and/or double-stranded RNA and/or DNA-based innate immune agonist.

In some aspects, the microneedle array comprises a first plurality of MOF particles comprising a first vaccine composition, and a second plurality of MOF particles comprising a second vaccine composition. The first vaccine composition may comprise a first antigen, and the second vaccine composition may comprise a second antigen. However, in other aspects, the first vaccine composition may comprise an antigen, while the second vaccine composition comprises an adjuvant.

In any aspects, each microneedle may further comprise a dissoluble biocompatible material, such as, for example, CMC, CMC and trehalose, CMC and sucrose, CMC and maltodextrin, CMC and lactose, hyaluronic acid, chitosan, alginate, gantrez, PVP, PVA, PVP and PVA, gelatin, poly lactic acid, pullulan, silk, polyphosphazene, poly-γ-glutamate, poly(lactic-co-glycolic acid), or a combination thereof.

Also disclosed herein are aspects of a method for fabricating a microneedle array comprising metal organic framework comprising vaccine components (antigens and adjuvants) disclosed herein. In some aspects, the method of fabrication comprises forming a solution comprising a dissoluble biocompatible material and a plurality of metal-organic framework (MOF) particles comprising a vaccine composition, and applying the solution to a microneedle array mold. However, in other aspects, the method comprises adding a solution comprising a dissoluble biocompatible material to a microneedle array mold that contains a plurality of metal-organic framework particles comprising a vaccine composition, to form the microneedle array. The method may further comprise loading the plurality of metal-organic framework particles comprising the vaccine composition into the mold before adding the solution comprising the dissoluble biocompatible material. Alternatively, the method may comprise loading into the mold precursors of the metal-organic framework and the vaccine composition to form the plurality of MOF particles comprising the vaccine composition in the mold before adding the solution comprising the dissoluble biocompatible material.

Additionally, aspects of a method of vaccinating a subject are disclosed herein. The method may comprise applying the microneedle array metal organic framework vaccine biocomposites disclosed herein to an area of the subject to deliver the vaccine composition to the subject.

The foregoing and other objects, features, and advantages of the disclosure will become more apparent from the following detailed description, which proceeds with reference to the accompanying figures.

### BRIEF DESCRIPTION OF THE DRAWINGS

FIG. 1 provides Transmission Electron Microscopy (TEM) images of pristine Zeolitic Imidazolate Framework-8 (ZIF-8) particles.

FIG. 2 provides Powder X-ray diffraction (PXRD) patterns of pristine ZIF-8 particles.

FIG. 3 is a graph of weight versus temperature, illustrating the thermogravimetric analysis of pristine ZIF-8 particles.

FIG. 4 is a graph of quantity absorbed versus relative pressure, illustrating the nitrogen absorption/desorption isotherm analysis of pristine ZIF-8 particles.

FIG. 5 is a graph of cumulative pore volume and change of pore volume versus pore width, illustrating the pore size distribution analysis of pristine ZIF-8 particles shows one pore measured at around 11 angstroms.

FIG. 6 is a schematic diagram illustrating that the pore size distribution matches the theoretical pore size of 12 angstroms.

FIGS. 7A-7C provide Transmission Electron Microscopy (TEM) images of pristine ZIF-8 particles having sizes of 66±20 nm (FIG. 7A), 198±38 nm (FIG. 7B), and 763±108 nm (FIG. 7C).

FIG. 8 provides Transmission Electron Microscopy (TEM) images of Ovalbumin (OVA) subunit vaccine-loaded ZIF-8 (OVA@ZIF-8) particles.

FIG. 9 provides powder X-ray diffraction (PXRD) patterns of Ovalbumin (OVA) subunit vaccine-loaded ZIF-8 (OVA-loaded ZIF-8) particles.

FIG. 10 is a graph of weight versus temperature, illustrating the thermogravimetric analysis of OVA-loaded ZIF-8 and unloaded ZIF-8 particles.

FIG. 11 is a graph of quantity absorbed versus relative pressure, illustrating the nitrogen absorption/desorption isotherm analysis of OVA-loaded ZIF-8 and unloaded ZIF-8 particles.

FIG. 12 provides digital images illustrating a hydrophobic material (Poly(dimethylsiloxane)—PDMS) piece wetted with 5 μl of OVA AF647 (top row) and 7.5 μl of OVA AF647@ZIF-8 bottom row).

FIG. 13 provides digital images illustrating a hydrophilic material (Agarose) wetted with 5 μl of OVA AF647 (top row) and 7.5 μl of OVA AF647@ZIF-8 bottom row).

FIG. 14 is a transmission electron microscopy (TEM) image of Alexa Fluor 647 labeled Ovalbumin vaccine-loaded zeolitic imidazolate frameworks-8 (OVA AF647@ZIF-8), with the size of 441±86 nm, with the vaccine loading efficiency of 99.82%, and with the vaccine loading of approximately 8%.

FIG. 15 is an optical microscopy image of the top view of OVA AF647@ZIF-8-loaded MNA production molds manufactured from plasma treated Poly(dimethylsiloxane) (PDMS).

FIG. 16 is an optical microscopy image of the view of OVA AF647@ZIF-8-loaded MNA production molds.

FIG. 17 is an optical stereomicroscopy image of obelisk-shaped Carboxymethylcellulose (CMC)/trehalose microneedles loaded with OVA AF647@ZIF-8. (CMC/Trehalose MNA-OVA AF647@ZIF-8 biocomposites).

FIG. 18 is an optical stereomicroscopy image of obelisk-shaped CMC/Trehalose microneedle-OVA AF647@ZIF-8 biocomposites.

FIG. 19 is a merged brightfield and fluorescent microscopy image of obelisk-shaped CMC/Trehalose microneedle-OVA AF647@ZIF-8 biocomposites.

FIG. 20 provides optical stereomicroscopy at different time points illustrating obelisk shaped CMC/Trehalose MNA-OVA AF647@ZIF-8 biocomposites applied to 4% agarose.

FIG. 21 provides optical stereomicroscopy at different time points illustrating obelisk shaped, OVA AF647-loaded CMC/Trehalose MNAs applied to 4% agarose.

FIG. 22 is an optical stereomicroscopy images of obelisk-shaped, carboxymethyl cellulose (CMC)/trehalose microneedle array-zeolitic imidazolate framework-8-Alexa Fluor 647 labeled Ovalbumin biocomposites (OVA AF647@ZIF-8@CMC/Trehalose MNA) after manufacturing.

FIG. 23 is an optical stereomicroscopy images of obelisk-shaped, carboxymethyl cellulose (CMC)/trehalose microneedle array-zeolitic imidazolate framework-8-Alexa Fluor 647 labeled Ovalbumin biocomposites (OVA AF647@ZIF-8@CMC/Trehalose MNA) after 20-minute application to mouse skin in vivo.

FIG. 24 is an in vivo live animal fluorescent imaging analysis of an OVA AF647@ZIF-8@CMC/Trehalose MNA biocomposite-treated mouse.

FIG. 25 is an optical stereomicroscopy image of obelisk-shaped, carboxymethyl cellulose (CMC)/trehalose microneedle array-zeolitic imidazolate framework-8-Alexa Fluor 647 labeled Ovalbumin biocomposites (OVA AF647@ZIF-8@CMC/Trehalose MNA) after manufacturing.

FIG. 26 is an optical stereomicroscopy image of the top surface of human skin explant after 5-minute treatment with OVA AF647@ZIF-8@CMC/Trehalose MNA biocomposites.

FIG. 27 is an optical stereomicroscopy image of remaining materials of OVA AF647@ZIF-8@CMC/Trehalose MNA biocomposites after 5-minute application to human skin sample.

FIG. 28 is a fluorescent imaging analysis of OVA AF647@ZIF-8@CMC/Trehalose MNA biocomposite-treated living human skin explant using an IVIS system.

FIG. 29 is an epifluorescence microscopy image of OVA AF647@ZIF-8@CMC/Trehalose MNA biocomposites-treated, cryosectioned human skin explant. Blue: DAPI and Red: OVA AF647@ZIF-8.

FIG. 30 is a schematic diagram illustrating the immunization schedule of mice showing that C57BL/6 mice received two doses of CMC/Trehalose MNA-delivered Ovalbumin (OVA) or OVA@ZIF-8 two weeks apart. 5 days after the booster dose, vaccine-induced humoral and cellular immune responses were evaluated by ELISA and in vivo lytic assay, respectively.

FIG. 31 is a graph of Anti-OVA IgG versus treatment, illustrating the serum levels of OVA-specific IgG antibodies (mean±SEM, N=3 mice per group).

FIG. 32 is a graph of percentage of specific cell lysis versus treatment, illustrating the activity of OVA-specific cytotoxic T lymphocytes (CTLs) for different treatment groups.

FIG. 33A is a transmission electron microscopy (TEM) image of Ovalbumin subunit protein vaccine loaded ZIF-8 (OVA@ZIF-8: Size: 441±86 nm and Concentration: 1000 μg/mL, Loading Efficiency: 99.82%).

FIG. 33B is a transmission electron microscopy (TEM) image of SARS-COV-2 Spike protein subunit vaccine loaded ZIF-8 (Spike protein@ZIF-8: Size: 318±66 nm and Concentration: 140 μg/mL).

FIG. 33C is a transmission electron microscopy (TEM) image of Adenovirus 5 loaded ZIF-8 (Ad5@ZIF-8: Size: 406±99 nm and Concentration: 7.8*1010 vp/mL).

FIG. 33D is a transmission electron microscopy (TEM) image of mRNA loaded ZIF-8. (mRNA@ZIF-8: Size: 454±107 nm and Concentration: 67 μg/mL, Loading Efficiency: 97.91%).

FIG. 34 provides powder X-ray diffraction (PXRD) patterns of ZIF-8 integrating various types of vaccines from FIGS. 33A-33D.

FIG. 35 provides graphs of frequency versus particle size, illustrating the size distribution of biodegradable zeolitic imidazolate framework-8 (ZIF-8) particles biomineralized with diverse biologics.

FIG. 36 is a graph of size versus vaccine-loaded MOF, comparing the sizes of biodegradable zeolitic imidazolate framework-8 (ZIF-8) particles biomineralized with diverse biologics.

FIG. 37 is an optical stereomicroscopy image of CMC/Trehalose MNAs integrating Alexa Fluor 647-labeled Ovalbumin (OVA)-loaded ZIF-8 (OVA AF647@ZIF-8).

FIG. 38 is a merged brightfield and fluorescent microscopy image of CMC/Trehalose MNAs integrating OVA AF647@ZIP-8.

FIG. 39 is an optical stereomicroscopy image of CMC/Trehalose MNAs incorporating SARS-COV-2 Spike subunit protein vaccine loaded ZIF-8 (S protein@ZIF-8).

FIG. 40 is a merged brightfield and fluorescent microscopy image of CMC/Trehalose MNAs integrating Alexa Fluor 647-labeled SARS-COV-2 S1 subunit vaccine-loaded ZIF-8 (S1 AF647@ZIF-8).

FIG. 41 is an optical microscope image illustrating CMC/Trehalose MNAs loaded with ZIF. 8 integrating Adenovirus 5 encoding enhanced green fluorescent protein (Ad5.eEGP@ZIF-8).

FIG. 42 is an optical microscope image illustrating CMC/Trebalose MNAs loaded with ZIF-8 incorporating mRNA vaccine encoding Luciferase (mRNA.Luciferase@ZIF-8).

FIG. 43 is an in vivo live animal fluorescent imaging analysis of SARS-COV-2 S1 AF647@ZIF-8@CMC/Trehalose MNA biocomposite-treated mouse.

FIG. 44 is a fluorescent imaging analysis of SARS-COV-2 S1 AF647@ZIF-8@CMC/Trehalose MNA biocomposite-treated living human skin explant using an IVIS system.

FIG. 45 is a brightfield microscopy image of OVA AF647@ZIF-8-Poly(I:C)Rhodamine@CMC/Trehalose biocomposites on PMMA stem.

FIG. 46 is a fluorescent microscopy image of OVA AF647@ZIF-8-Poly(I:C)Rhodamine@CMC/Trehalose biocomposites on PMMA stem with the filter corresponding to Rhodamine.

FIG. 47 is a fluorescent microscopy image of OVA AF647@ZIF-8-Poly(I:C)Rhodamine@CMC/Trehalose biocomposites on PMMA stem with the filter corresponding to AF647.

FIG. 48 is a merged microscopy image of FIGS. 45-47.

FIG. 49 is an optical stereomicroscopy image of OVA AF647@ZIF-8-Poly(I:C)Rhodamine@CMC/Trehalose biocomposites on PMMA stem after skin application.

FIG. 50 is brightfield microscopy image of OVA AF647@ZIF-8-Poly(I:C)Rhodamine@CMC/Trehalose biocomposites on PMMA stem after skin application.

FIG. 51 is an in vivo live animal fluorescent imaging analysis of OVA AF647@ZIF-8-Poly(I:C)Rhodamine@CMC/Trehalose on PMMA stem MNA biocomposite-treated mouse using an IVIS system illustrating the rhodamine fluorescence.

FIG. 52 is an in vivo live animal fluorescent imaging analysis of OVA AF647@ZIF-8-Poly(I:C)Rhodamine@CMC/Trehalose on PMMA stem MNA biocomposite-treated mouse using an IVIS system illustrating the AF647 fluorescence.

FIG. 53 is a fluorescent imaging analysis of OVA AF647@ZIF-8-Poly(I:C) Rhodamine@CMC/Trehalose on PMMA stem MNA biocomposite-treated living human skin explant using an IVIS system illustrating the rhodamine fluorescence.

FIG. 54 is a fluorescent imaging analysis of OVA AF647@ZIF-8-Poly(I:C) Rhodamine@CMC/Trehalose on PMMA stem MNA biocomposite-treated living human skin explant using an IVIS system illustrating the AF647 fluorescence.

FIG. 55A is a fluorescent microscopy images of DC 2.4 cells transfected with mRNA.mCherry recovered from mRNA@ZIF-8.

FIG. 55B is flow cytometry analysis of DC 2.4 cells transfected with mRNA.mCherry recovered from mRNA@ZIF-8.

FIG. 56 is a graph of Anti-SARS-COV-2 S1 IgG versus treatment, illustrating the serum levels of SARS-COV-2-S1-specific IgG antibodies (N=5 mice per group).

FIG. 57A is a scanning electron microscopy (SEM) image of Hendra virus soluble glycoprotein (GP)-loaded ZIF-8 (HeV-sG@ZIF-8).

FIG. 57B is a scanning electron microscopy (SEM) image of Hendra virus soluble glycoprotein (GP)+ Poly(I:C)-loaded ZIF-8 (HeV-sG+Poly(I:C) @ZIF-8).

FIG. 57C is a graph of Anti-HeV-sG IgG versus treatment, illustrating the serum levels of HeV-sG-specific IgG antibodies (N=5 mice per group).

FIG. 58A is a set of representative photographs of murine skin treated with HeVsG+Poly(I:C) @ZIF-8-loaded MNAs.

FIG. 58B is a set of measurements of body weight of mice treated with HeVsG+Poly(I:C)@ZIF-8-loaded MNAs (N=5 mice).

FIG. 58C is a set of measurements of body temperature of mice treated with HeVsG+Poly(I:C) @ZIF-8-loaded MNAs (N=5 mice).

### DETAILED DESCRIPTION

### I. Definitions and Terms

The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.”

Unless otherwise indicated, all numbers expressing quantities of components, molecular weights, percentages, temperatures, times, and so forth, as used in the specification or claims, are to be understood as being modified by the term “about.” Unless context indicated otherwise, “about” refers to plus or minus 5% of a reference value. For example, “about” 100 refers to 95 to 105.

Unless explained otherwise, all technical and scientific terms are used according to conventional usage. Although methods and materials similar or equivalent to those described herein can be used in the practice or testing of the present disclosure, suitable methods and materials are described below. The materials, methods, and examples are illustrative only and not intended to be limiting.

The term “adjuvant” refers to any component added to a vaccine that modifies the effect of the vaccine, such as enhancing an immune response and/or stabilizing a formulation. Adjuvants are often pharmacological and/or immunological agents. In some aspects, the adjuvant is a small-molecule immune potentiator, such as saponin; a STING pathway agonist; or a nucleic acid and nucleic acid origami immune potentiator, such as a single-stranded and/or double-stranded RNA and/or DNA-based innate immune agonist.

The term “effective amount” refers to an amount of a vaccine composition sufficient to provide a desired result, such as provide or enhance an immune response and/or provide protection from a pathogen. It is understood that to obtain a protective immune response against a pathogen of interest can require multiple administrations of a disclosed vaccine composition, and/or administration of a disclosed vaccine composition followed or preceded by administration of alternate vaccine compositions targeted to the pathogen. Thus, the disclosed vaccine composition can be used as the “prime” or “boost” (or both) component of a prime-boost immunization protocol. The amount of a vaccine composition which constitutes an “effective amount” will vary depending on the vaccine composition, the disease, the nature and age of the subject, and the like.

The term “dissolvable microneedle” refers to micron-scale needles manufactured from water-soluble or degradable biomaterials, or micron-scale needles fabricated from water-soluble or degradable biomaterials integrated with non-dissolvable and non-degradable biocompatible material-based stem regions.

The terms “subject” or “patient” refer to mammals and other animals, particularly humans. Thus, disclosed technology is applicable to both human therapy and veterinary applications.

### II. Overview

Dissolvable microneedle arrays enable efficient and safe vaccine delivery to the targeted skin microenvironments and mucosal surfaces. However, vaccines incorporated into dissolvable microneedle arrays can be adversely affected by chemical and thermal, biological, and environment stressors during the manufacturing, skin delivery (e.g., extracellular enzymes), and storage and distribution stages, respectively. Thus, each vaccine typically requires a specific formulation to achieve thermal, chemical, and/or biological stability. This can increase the cost and complexity of preparing a vaccine formulation. Disclosed herein are aspects of a metal-organic framework (MOF)-vaccine biocomposite that is suitable for use in a dissolvable microneedle array. The MOF provides the chemical and thermal stability of the vaccine during manufacturing, storage, and distribution of dissolvable microneedle arrays. Further, the MOF provides protection for the vaccine in the extracellular space, but decomposes at the proper cellular locations to release the vaccine. Importantly, the MOF provides a standardized delivery vehicle for vaccine components that is suitable for use with a wide variety of vaccine types, including, but not limited to, protein vaccines, polysaccharide vaccines, mRNA vaccines, self-replicating mRNA vaccines, nucleic acid vaccines, inactivated virus vaccine, cell lysate vaccine, viral vector vaccines, and bacterial vaccines, as well as for a variety of adjuvant types, including, but not limited to, nucleic acid adjuvants and small-molecule adjuvants.

### III. MOF-Vaccine Biocomposite

The MOF in the biocomposite may be any MOF suitable for use in a microneedle array and suitable for use with a vaccine composition. Exemplary MOFs include but are not limited to zeolitic imidazolate frameworks (ZIFs), such as ZIF-8, ZIF-10, ZIF-90, iron-based MOFs, such as, MIL-53 and MIL-88, copper-based MOFs, such as HKUST-1, and other MOFs, such as MAF-7, Eu/Tb-BDC, magnesium-based MOFs, chromium-based MOFs, calcium-based MOFs, europium-based MOFs, bismuth-based MOFs, titanium-based MOFs, cobalt-based MOFs, nickel-based MOFs, or zirconium-based MOFs.

In some aspects, the MOF is selected to have reaction synthesis conditions that are compatible with the vaccine composition that is contained within the MOF. Suitable reaction conditions include, but are not limited to, a reaction temperature, solvent, and/or reagent(s) that are compatible with the vaccine composition such that the vaccine composition does not substantially degrade during MOF formation.

In some aspects, the MOF is selected to be stable in the extracellular space but to dissolve once the MOF enter a cell. In some aspects, the MOF dissolves in an acidic pH environment, such as a pH of less than 7 or 6.5 or less, for example in an environment having a pH of from less than 7 to 5 or less, such as from 6.5 or less to 5 or less.

The MOF-vaccine biocomposite also comprises one or more vaccine components. In some aspects, the vaccine component is an antigen, and may be a protein, mRNA, self-replicating mRNA, nucleic acid antigen, recombinant viral vectored antigen, nucleic acid origami antigen, extracellular vesicle antigen, inactivated virus antigen, liposome encapsulated antigen, extracellular vesicle encapsulated antigen, cell lysate antigen, bacterial antigen, or a combination thereof. In some aspects, multiple antigens are encapsulated into the same MOF. In some aspects, different antigens are encapsulated into different MOFs.

In some aspects, the vaccine antigen is from a pathogen, including viruses, parasites, fungi and bacteria. In some aspects, the pathogen is a virus, such as, but not limited to a virus from one of the following families: Retroviridae (for example, human immunodeficiency virus (HIV); human T-cell leukemia viruses (HTLV); Picornaviridae (for example, polio virus, hepatitis A virus; hepatitis C virus; enteroviruses, human coxsackie viruses, rhinoviruses, echoviruses; foot-and-mouth disease virus); Calciviridae (such as strains that cause gastroenteritis); Togaviridae (for example, equine encephalitis viruses, rubella viruses); Flaviridae (for example, dengue viruses; yellow fever viruses; West Nile virus; St. Louis encephalitis virus; Japanese encephalitis virus; and other encephalitis viruses); Coronaviridae (for example, coronaviruses; severe acute respiratory syndrome (SARS) virus; Rhabdoviridae (for example, vesicular stomatitis viruses, rabies viruses); Filoviridae (for example, Ebola viruses); Paramyxoviridae (for example, parainfluenza viruses, mumps virus, measles virus, respiratory syncytial virus (RSV)); Orthomyxoviridae (for example, influenza viruses); Bunyaviridae (for example, Hantaan viruses; Sin Nombre virus, Rift Valley fever virus; bunya viruses, phleboviruses and Nairo viruses); Arena viridae (hemorrhagic fever viruses; Machupo virus; Junin virus); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses; BK-virus); Adenoviridae (most adenoviruses); Herpesviridae (herpes simplex virus (HSV)-1 and HSV-2; cytomegalovirus (CMV); Epstein-Barr virus (EBV); varicella zoster virus (VZV); and other herpes viruses, including HSV-6); Poxviridae (variola viruses, vaccinia viruses, pox viruses); and Iridoviridae (such as African swine fever virus); Filoviridae (for example, Ebola virus; Marburg virus); Caliciviridae (for example, Norwalk viruses) and unclassified viruses (for example, the etiological agents of Spongiform encephalopathies, the agent of delta hepatitis (thought to be a defective satellite of hepatitis B virus); and astroviruses).

In other aspects, the target antigen is an antigen from a bacteria, such as, but not limited to, Helicobacter pyloris, Borelia burgdorferi, Legionella pneumophilia, Mycobacteria sps (such as. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, corynebacterium sp., Erysipelothrix rhusiopathiae, Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, or Actinomyces israelli.

In further aspects, the antigen is from a fungus, such as Cryptococcus neoformans, Histoplasma capsulatum, Coccidioides immitis, Blastomyces dermatitidis, Chlamydia trachomatis, or Candida albicans. In other aspects, antigen is from a parasite, such as, but not limited to, Plasmodium falciparum or Toxoplasma gondii.

In some aspects, the antigen is a cancer antigen. The cancer can be a solid tumor or a hematogenous cancer. In particular examples, the solid tumor is a sarcoma or a carcinoma, such as fibrosarcoma, myxosarcoma, liposarcoma, chondrosarcoma, osteogenic sarcoma, or another sarcoma, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, or a CNS tumor (such as a glioma, astrocytoma, medulloblastoma, craniopharyogioma, ependymoma, pinealoma, hemangioblastoma, acoustic neuroma, oligodendroglioma, menangioma, melanoma, neuroblastoma or retinoblastoma).

In some aspects, the hematogenous cancer is a leukemia, such as an acute leukemia (such as acute lymphocytic leukemia, acute myelocytic leukemia, acute myelogenous leukemia and myeloblastic, promyelocytic, myelomonocytic, monocytic and erythroleukemia); a chronic leukemia (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia or myelodysplasia.

Tumor antigens are known in the art and include, for example, carcinoembryonic antigen (CEA), human chorionic gonadotropin (HCG), alpha-fetoprotein (AFP), lectin-reactive AFP, (AFP-L3), thyroglobulin, RAGE-1, MN-CA IX, human telomerase reverse transcriptase (hTERT), RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), melanoma-associated antigen (MAGE), ELF2M, neutrophil elastase, ephrinB2 and CD22. The CH2 or CH3 domain molecules can also bind any cancer-related proteins, such IGF-I, IGF-II, IGR-IR or mesothelin.

In some aspects, the antigen is a self-antigen. The antigen can be an antigen associated with an autoimmune disease, such as rheumatoid arthritis, juvenile oligoarthritis, collagen-induced arthritis, adjuvant-induced arthritis, Sjögren's syndrome, multiple sclerosis, experimental autoimmune encephalomyelitis, inflammatory bowel disease (for example, Crohn's disease, ulcerative colitis), autoimmune gastric atrophy, pemphigus vulgaris, psoriasis, vitiligo, type 1 diabetes, non-obese diabetes, myasthenia gravis, Grave's disease, Hashimoto's thyroiditis, sclerosing cholangitis, sclerosing sialadenitis, systemic lupus erythematosis, autoimmune thrombocytopenia purpura, Goodpasture's syndrome, Addison's disease, systemic sclerosis, polymyositis, dermatomyositis, autoimmune hemolytic anemia or pernicious anemia.

Additionally, or alternatively, the vaccine component may comprise an adjuvant, which may be a single-stranded RNA, double-stranded RNA, single-stranded DNA, synthetic polynucleotides, liposomes, extracellular vesicles, aluminum salts, bacterial lipopolysaccharides, Polyphosphazanes, STING, TLR, CLR, and RLR agonists. In some aspects, adjuvants are encapsulated into the MOF that also encapsulates the antigen. In some aspects, adjuvants are absorbed onto the surface of the MOF that encapsulates the antigen. In some aspects, adjuvants are encapsulated into the MOF that is different from the MOF encapsulating the antigen. In some aspects, adjuvants are absorbed onto the MOF that is different from the MOF encapsulating the antigen.

Usually, the pores of the MOF are too small to allow the vaccine component(s) to enter or exit the MOF structure. Accordingly, the MOF typically is synthesized in the presence of the vaccine component(s) such that the MOF may nucleate around the vaccine component(s), thereby encapsulating the component(s) in the MOF.

The MOF-vaccine biocomposite forms as particles, such as nanoparticles or microparticles. In some examples, the MOF-vaccine biocomposite particles have an average size of from 10 nm to 50,000 nm, such as from 10 nm to 10,000 nm, from 10 nm to 5,000 nm, from 10 nm to 2000 nm, from 10 nm to 100 nm, from 200 nm to 900 nm, or from 1000 nm to 2000 nm. As used herein, the size of the particle refers to the longest dimension of the particle. In some aspects, the MOF with larger (i.e., larger than the size of vaccine components) pore sizes are synthesized and the vaccine components can be loaded post-synthesis.

### IV. Microneedle Array Comprising the MOF-Vaccine Biocomposite

Disclosed herein are aspects of microneedle arrays comprising the MOF-vaccine biocomposite particles. In some aspects, the microneedle array comprises a dissolvable biocompatible material that forms the needles and comprises the MOF-vaccine biocomposite particles. In some aspects, the MOF particles are located within and/or on the surface of the needles. Dissolvable biocompatible materials are non-toxic and dissolve or degrade under the physiological conditions of the skin. Suitable dissolvable biocompatible materials include, but are not limited to, carboxymethylcellulose (CMC), CMC and trehalose, CMC and sucrose, CMC and maltodextrin, CMC and lactose, hyaluronic acid, chitosan, alginate, gantrez, polyethylene glycol (PEG), polyvinylpyrrolidone (PVP), poly(vinyl alcohol) (PVA), PVP and PVA, gelatin, poly lactic acid, pullulan, silk, polyphosphazene, poly-γ-glutamate, poly(lactic-co-glycolic acid), or combinations thereof.

In some aspects, the dissolvable material is selected to at least partially dissolve in the skin microenvironment or in the mucosal tissues to release at least a portion of the MOF-vaccine biocomposite.

In some aspects, the microneedles in the array have a size and shape suitable to facilitate vaccine delivery to a subject.

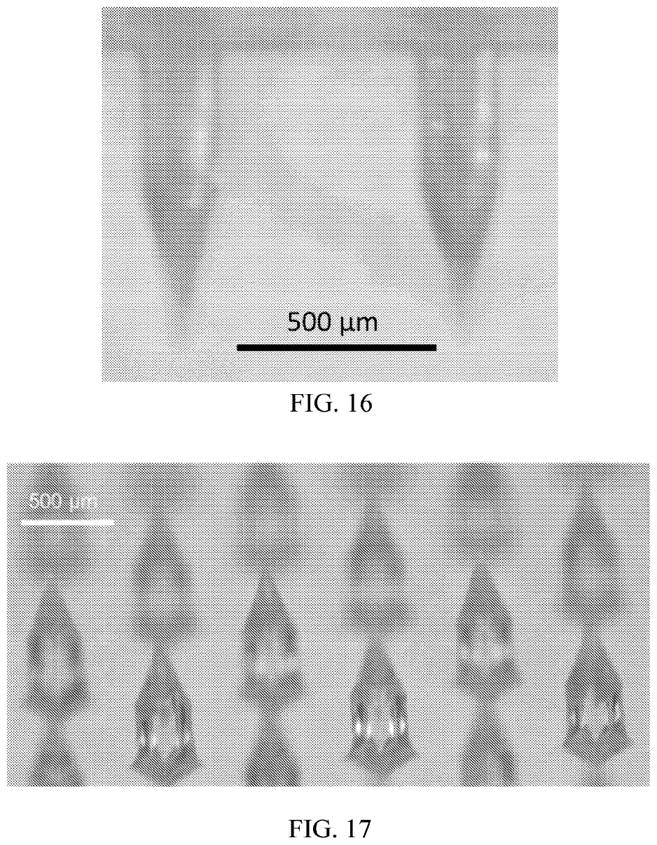

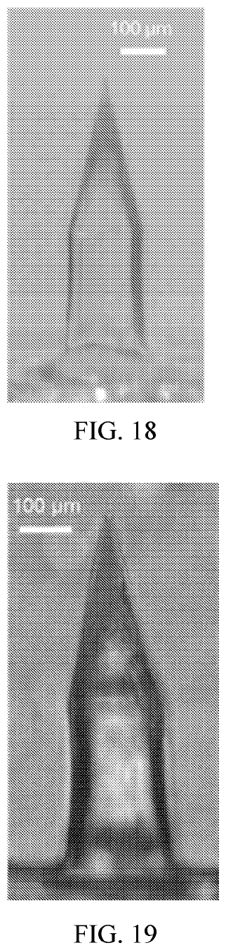

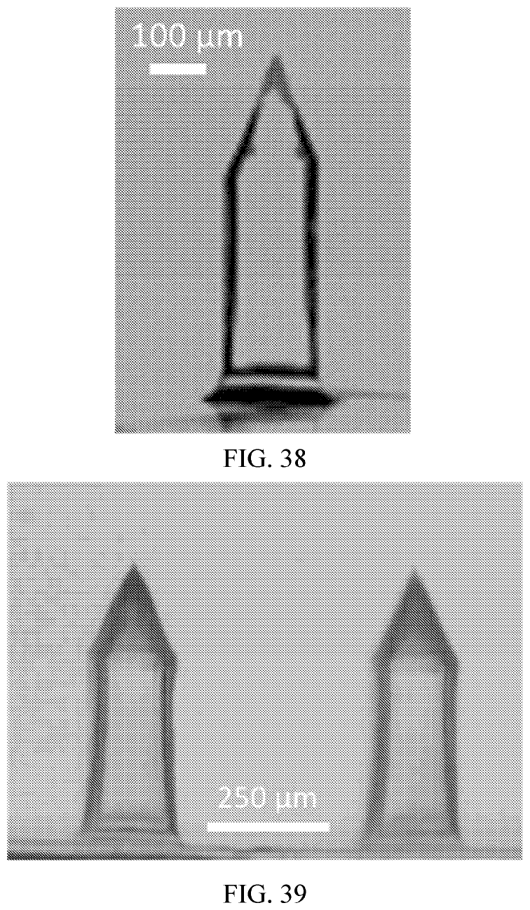

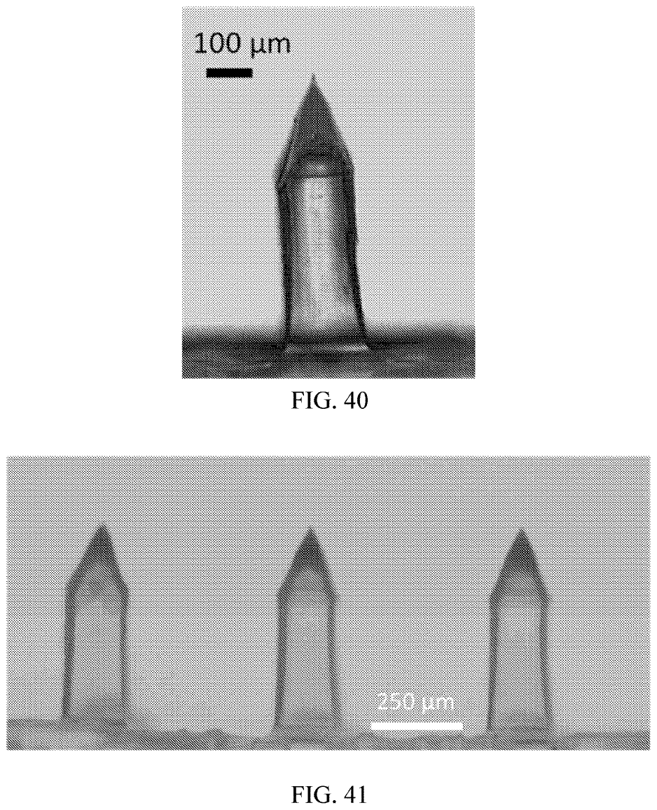

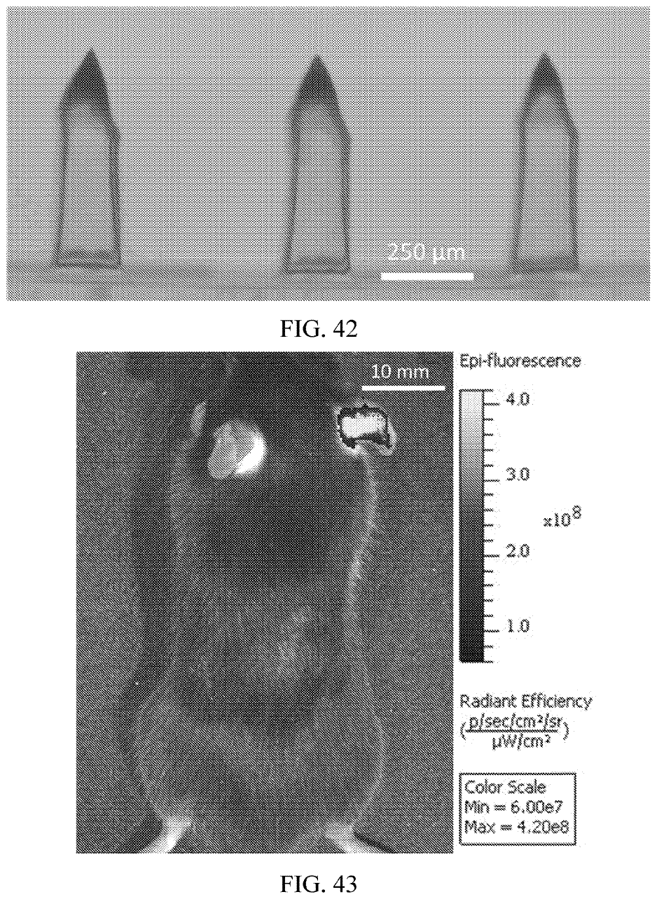

In some aspects, the microneedle array comprises microneedles having a length (height from a base layer to the tip) of from 50 μm to 1000 μm, such as from 300 μm to 750 μm. In some aspects, the base layer lacks the MOF particles comprising the vaccine composition. In some aspects, the microneedle has a base diameter or width of from 50 μm to 500 μm and tapers to a point. In some aspects, the taper starts at the base, thereby forming a pyramidal or cone shaped needle. But in other aspects, the taper starts part way up the microneedle, such as at about 20%, about 30%, about 40%, about 50%, about 60% or about 70% along the microneedle from the base. In such examples, the microneedles comprise a pillar structure extending from the base, and the tapered portion of the microneedle forms a pyramidal or cone shaped structure located on the end of the pillar structure. See, for example, FIGS. 15-18, 21 and 37-42. And as viewed from above, the microneedles may have a substantially circular shape, or they may have a rectangular, square, triangular, trapezoidal, polygonal, or irregular shape.

In some aspects, the microneedle array has a sufficient number of needles to provide a beneficial amount of the vaccine composition to a patient. In some aspects, the microneedle array has from 5 needle to 1000 needles per (5 mm to 30 mm)×(5 mm to 30 mm) array, such as from 50 needles to 150 needles per 10 mm×10 mm array, from 100 needles to 400 needles per 20 mm×20 mm array, from 250 needles to 750 needles per 25 mm×25 mm array, or about 1000 needles per 30 mm×30 mm array.

In the array, the microneedles are separated from each other by a distance suitable to facilitate delivery of an effective amount of the vaccine to the subject. In some aspects, the microneedles are arranged in a regular pattern, such as in parallel and perpendicular lines (FIG. 15). In some aspects, the microneedles are arranged such that a gap between two needles is from 100 μm to 1000 μm, such as from 250 μm to 750 μm.

In some aspects, the MOF-vaccine biocomposite is located throughout the microneedles, but in other aspects, the MOF-vaccine biocomposite is substantially located in the tapered portion of the microneedles. In some aspects, greater than 50% of the MOF-vaccine biocomposite is located in the tapered portion of the microneedle, such as greater than 60%, greater than 70%, greater than 80%, greater than 90%, greater than 95%, greater than 97% or greater than 99% of the MOF-vaccine biocomposite (FIGS. 17, 18 and 19).

### V. Master Mold and Production Mold Preparation

A master mold of the microneedle array-MOF-vaccine biocomposite is prepared using microfabrication strategies, including but not limited to, 3D laser printing, photolithography, dry etching, or micromachining, to dictate microneedle and array geometries, as well as the dimensions of microneedles and their spatial distribution across the array.

For example, microneedle arrays can be fabricated based on a master mold (positive) to production mold (negative) to array (positive) methodology. Micromilling technology can be used to generate various micro-scale geometries on a variety of materials, including metal, polymer, and ceramic parts. Micromilled master molds of various shapes and configurations can be effectively used to generate multiple identical female production molds. The female production molds can then be used to microcast various microneedle arrays. In some aspects, the female production molds can be utilized to replicate master molds using UV-curable resins.

The master mold is desirably formed of a material that is capable of being reused so that a single mastermold can be repeatedly used to fabricate a large number of production molds. Similarly, each production mold is desirably able to fabricate multiple microneedle arrays.

Master molds can be micromilled from various materials, including, for example, stainless steel, brass, aluminum, PEEK, PMMA, Cirlex® (DuPont, Kapton® polyimide). The master mold material preferably is able to be cleanly separated from the production mold material and preferably is able to withstand any heighted curing temperatures that may be necessary to cure the production mold material. For example, in an illustrated aspect, the silicone-based compound SYLGARD® 184 (Dow Corning) is the production mold material and that material may require a curing temperature of about 80-90 degrees Celsius.

Alternatively, a master mold may be prepared using 3D laser printing. The master mold may be fabricated from IP-S photoresist by 3D direct laser writing. IP-S is a specific material designed for 3D laser lithography and provides high resolution and mechanical integrity for micro- and nano-structures. 3D laser lithography based on two-photon polymerization provides an effective means for fabricating microneedle array designs with smooth edges and sharp tips and without any unwanted residues (e.g., machining chips).

Additional information concerning preparing a master mold can be found in United States patent application publication Nos. 2016/0136407 and 2022/0241570, both of which are incorporated herein by reference in their entirety.

Master molds can be used to fabricate flexible production molds from a suitable material, such as EcoFlex (hydrophobic), polydimethylsiloxane (PDMS, hydrophobic), plasma-treated PDMS (more hydrophilic than PDMS), a copolymer of PDMS and poly(ethyleneglycol) (PEG) (more hydrophilic than PDMS), and Agarose (hydrophilic). In some aspects, plasma treated PDMS or PDMS-PEG is used.

In some aspects, the material for the production mold is selected to have surface properties that are compatible with the MOF and/or vaccine composition. The surface properties include, but are not limited to, surface interactions between the mold material and the MOF and/or vaccine composition, such as hydrophobic and/or hydrophilic interactions. In some aspects, the production mold material is selected to have a desired hydrophobicity or hydrophilicity to localize the vaccine-loaded MOFs in the microneedle-shaped wells of microneedle array molds.

Additionally, in aspects where the MOF-vaccine biocomposite is prepared in the mold, the mold material may be selected to reduce interactions between the vaccine composition and the mold material before the MOF-vaccine biocomposite is formed.

### VI. Manufacturing the Microneedle Array-MOF-Vaccine Biocomposite

In some aspects, the microneedle array comprising a MOF-vaccine biocomposite is prepared by first forming the MOF-vaccine biocomposite and then forming the microneedle array comprising the MOF-vaccine biocomposite. In some aspects, the MOF-vaccine biocomposite are prepared outside of the microneedle array mold. In such aspects, a solution is formed comprising the MOF starting materials and the vaccine composition. The solution is formed in a aqueous solvent, such as water. The solution is agitated, such as by stirring, shaking and/or vortexing, and after forming, the MOF-vaccine biocomposite is separated from the solution. The MOF-vaccine is then resuspended in a suitable suspension liquid and the suspension is added to the production mold. The suspension liquid may comprise water-soluble hydrophilic biomaterials and/or surfactants, such as carboxymethyl cellulose (CMC), CMC and trehalose, CMC and sucrose, CMC and maltodextrin, CMC and lactose, hyaluronic acid, chitosan, alginate, gantrez, polyvinylpyrrolidone (PVP), polyvinyl alcohol (PVA), PVP and PVA), PEG, PVP, PVA, PVP and PVA, gelatin, pullulan, silk, polyphosphazene, poly-γ-glutamate and/or nucleic acid adjuvants (Poly(I:C) or CpG). In some aspects, the suspension liquid may be selected to coat the MOF-vaccine biocomposite particles with hydrophilic compounds to overcome any hydrophobic/hydrophobic interaction and/or charge interactions with the production molds.

Additionally, or alternatively, the suspension liquid may comprise an organic solvent which may be a water soluble, such as an alcohol (for example, methanol, ethanol, isopropanol, for a combination thereof), acetonitrile, or a combination thereof.

After the MOF-vaccine biocomposite has been added to the mold, the dissolvable/structural material of the array is added to form the microneedles and the backing layer.

In alternative aspects, the MOF components and the vaccine composition are added directly to the production mold and the MOF-vaccine biocomposite forms in situ. Once the MOF-vaccine biocomposite has formed, the dissolvable/structural material of the array is added to form the microneedles and the backing layer.

In some aspects, the microneedle array comprises microneedles having a length (height from a base layer to the tip) of from 50 μm to 1000 μm, such as from 300 μm to 750 μm. In some aspects, the base layer lacks the MOF particles comprising the vaccine composition. In some aspects, the microneedle has a base diameter or width of from 50 μm to 500 μm and tapers to a point to form the tip of the needle. In some aspects, the taper starts at the base, thereby forming a pyramidal or cone shaped needle. But in other aspects, the taper starts part way up the microneedle, such as at about 20%, about 30%, about 40%, about 50%, about 60% or about 70% along the microneedle from the base. In such examples, the microneedles comprise a pillar structure extending from the base, and the tapered portion of the microneedle forms a pyramidal or cone shaped structure located on the end of the pillar structure. And as viewed from above, the microneedles may have a substantially circular shape, or they may have a rectangular, square, triangular, trapezoidal, polygonal, or irregular shape.

In some aspects, the microneedle array has a sufficient number of needles to provide a beneficial amount of the vaccine composition to a patient. In some aspects, the microneedle array has from 5 needle to 1000 needles per (5 mm to 30 mm)×(5 mm to 30 mm) array, such as from 50 needles to 150 needles per 10 mm×10 mm array, from 100 needles to 400 needles per 20 mm×20 mm array, from 250 needles to 750 needles per 25 mm×25 mm array, or about 1000 needles per 30 mm×30 mm array.

In the array, the microneedles are separated from each other by a distance suitable to facilitate delivery of an effective amount of the vaccine to the subject. In some aspects, the microneedles are arranged in a regular pattern, such as in parallel and perpendicular lines (FIG. 15). In some aspects, the microneedles are arranged such that a gap between two needles is from 100 μm to 1000 μm, such as from 250 μm to 750 μm.

In some aspects, the MOF-vaccine biocomposite is located throughout the microneedles, but in other aspects, the MOF-vaccine biocomposite is substantially located in the tapered portion of the microneedles. In some aspects, greater than 50% of the MOF-vaccine biocomposite is located in the tapered portion of the microneedle, such as greater than 60%, greater than 70%, greater than 80%, greater than 90%, greater than 95%, greater than 97% or greater than 99% of the MOF-vaccine biocomposite.

### VII. Use of the Microneedle Array-MOF-Vaccine Biocomposite

The disclosed microneedle array-MOF-vaccine biocomposite is useful for delivering a vaccine to a subject in need thereof. Typically, the microneedle array is applied to a location on the skin of the subject, such as in the form of a patch. Suitable locations on the patent include, but are not limited to, an arm, leg, chest, back, or neck of the subject, or mucosal surfaces. The patch is applied for a time period suitable to facilitate delivery of an effective amount of the MOF-vaccine biocomposite. In some aspects, the patch is applied to the skin for from greater than zero to 24 hours, such as from 1 hour to 2 hours, from 1 minute to 30 minutes.

### VIII. EXAMPLES

### Example 1

### Synthesis of Zeolitic Imidazolate Framework-8 (ZIF-8)

A 3M solution of 2-methylimidazole was prepared by sonicating a mixture of 2-methylimidazole (246.3 mg, 3 mmol) in 1 mL nuclease-free water (H₂O). A 1M solution of zinc acetate dihydrate was prepared by sonicating a mixture of zinc acetate dihydrate (183.5 mg, 1 mmol) in 1 mL nuclease-free water. 3M 2-methylimidazole solution (853 μL, 2.56 mmol) and nuclease-free water (67 μl) were added to a 1.5 mL Eppendorf microcentrifuge tube in sequence and the tube was vortexed for 10 seconds. 1M zinc acetate dihydrate (80 μL, 0.08 mmol) was added to the tube that was vortexed again for 30 seconds. The solution reacted at room temperature or at lower temperature for 1 hour or lesser period of time to yield a turbid solution. The suspension was centrifuged at 10,000 rpm for 2 minutes. The precipitate was washed with nuclease-free water (2 mL, 2×) and ethanol or methanol (2 mL, 2×) and dried under vacuum to obtain pristine ZIF-8 crystals as a white or colored (in the case of fluorescent labeled biocargos) precipitate.

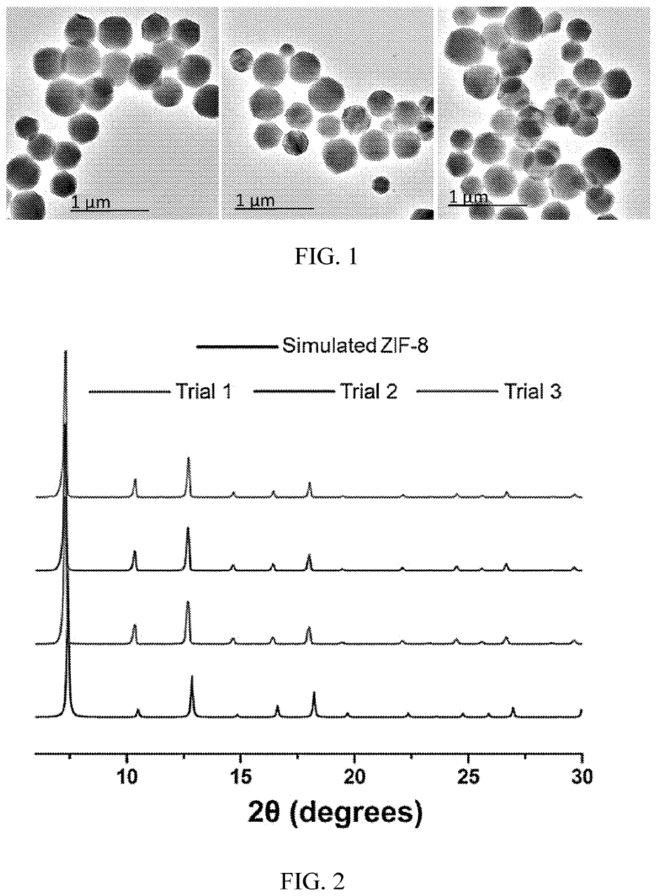

Transmission electron microscopy (TEM, A JEOL JEM2100F) analysis showed successful formation of polydisperse ZIF-8 particles with an average size distribution of 468±92 nm (104 ZIF-8 particles were measured) (FIG. 1).

Powder X-ray diffraction (PXRD, A Bruker AXS D8 Discover X-Ray Powder Diffractometer) analysis suggested that PXRD patterns of ZIF-8 particles showed patterns that closely matched the simulated results, thereby indicating that the successfully synthesized ZIF-8 particles were highly crystalline and phase pure (FIG. 2).

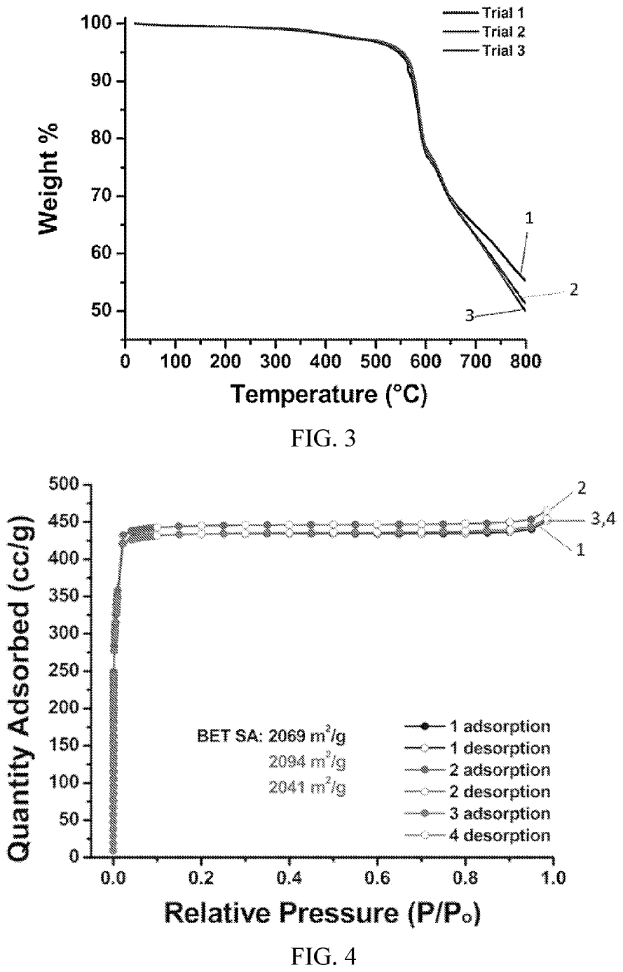

Thermogravimetric analysis (TGA, TA Instruments Q500 Thermal Analysis System) demonstrated thermal stability and degradation profile of ZIF-8, and suggested that pristine ZIF-8 particles were thermally stable until 550° C. at which the ZIF-8 particles started to decompose (FIG. 3).

Nitrogen adsorption/desorption isotherms (Micromeritics 3Flex Gas Absorption Analyzer) indicated that pristine ZIF-8 particles were highly porous with a Type-I Isotherm and with a Brunauer-Emmett-Teller (BET) surface area of approximately 2,070 m²/g. Analysis of multiple ZIF-8 samples using the aforementioned techniques demonstrated the reproducibility of the facile, scalable MOF synthesis protocol (FIG. 4).

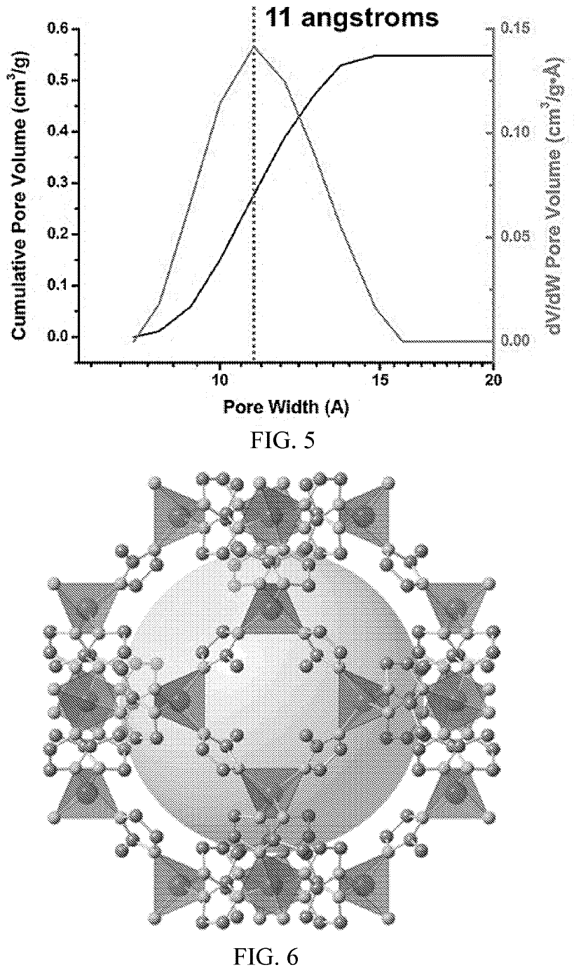

The pore size distribution analysis showed pore sizes around 11 angstroms (1.1 nm), which closely matched the theoretical pore size of 12 angstroms (FIGS. 5 and 6). The relative sizes of vaccine antigens/adjuvants and ZIF-8 pores suggested that vaccines may be large to be loaded within the pore network of ZIF-8. However, vaccine components could be loaded within the mesopores within the ZIF-8 bulk crystals via biomimetic mineralization.

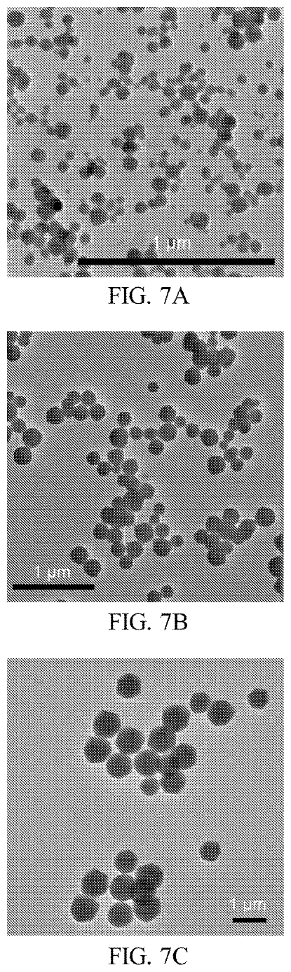

Transmission electron microscopy (TEM, A JEOL JEM2100F) analysis showed successful formation of ZIF-8 particles with tunable sizes (FIG. 7A: 66±20 nm, FIG. 7B: 198±38 nm, and FIG. 7C: 763±108 nm).

### Example 2

### Synthesis of Vaccine-Loaded Zeolitic Imidazolate Framework-8 (ZIF-8)

A 3M solution of 2-Methylimidazole was prepared by sonicating a mixture of 2-methylimidazole (246.3 mg, 3 mmol) in 1 mL nuclease-free water (H₂O). A 1M solution of zinc acetate dihydrate was prepared by sonicating a mixture of zinc acetate dihydrate (183.5 mg, 1 mmol) in 1 mL nuclease-free water. An Ovalbumin (OVA) vaccine solution was prepared by mixing 25.6 mg OVA with 1 mL nuclease-free water. 3M 2-methylimidazole solution (853 μL, 2.56 mmol), nuclease-free water (28 μl), and OVA solution (39 μl, 1 mg) were added to a 1.5 mL Eppendorf microcentrifuge tube in sequence and the tube was vortexed for 10 seconds. Then 1M zinc acetate dihydrate (80 μL, 0.08 mmol) was added, and the tube was vortexed again for 30 seconds. The solution reacted at room temperature or at lower temperature for 1 hour or lesser period of time to yield a turbid solution. The suspension was centrifuged at 10,000 rpm for 2 minutes. The precipitate was washed with nuclease-free water (2 mL, 2×) and ethanol or methanol (2 mL, 2×) and dried under vacuum to obtain subunit vaccine@ZIF-8 crystals as a precipitate.

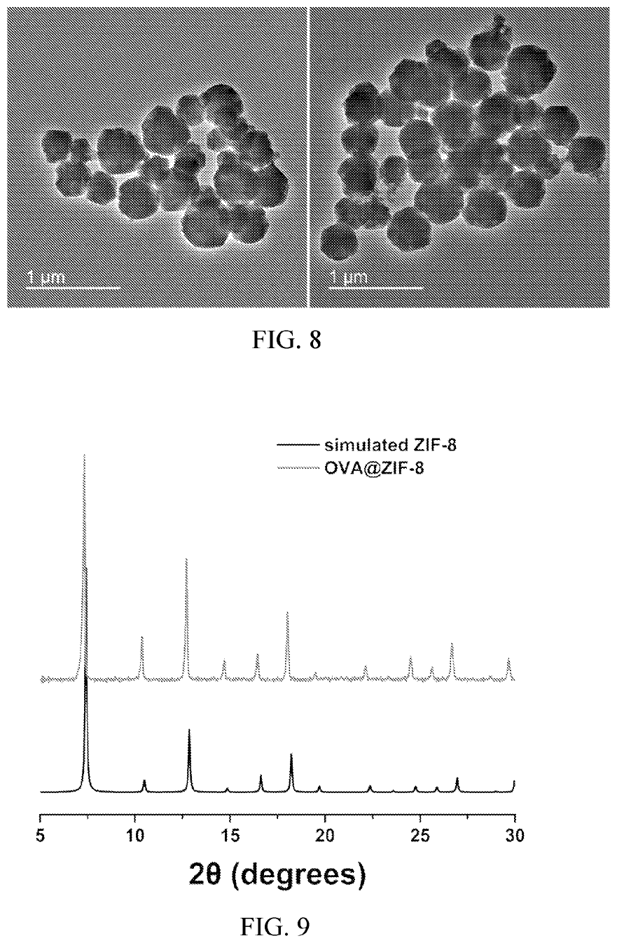

Transmission electron microscopy (TEM, A JEOL JEM2100F) analysis showed successful formation of vaccine-loaded ZIF-8 particles with an average size distribution of 441±86 nm (101 OVA@ZIF-8 particles were measured) (FIG. 8). There is no significant difference between the size of pristine ZIF-8 and that of OVA@ZIF-8.

Powder X-ray diffraction (PXRD, A Bruker AXS D8 Discover X-Ray Powder Diffractometer) analysis showed that the patterns of OVA@ZIF-8 matched the simulated results, indicating that OVA@ZIF-8 particles were highly crystalline and phase pure (FIG. 9).

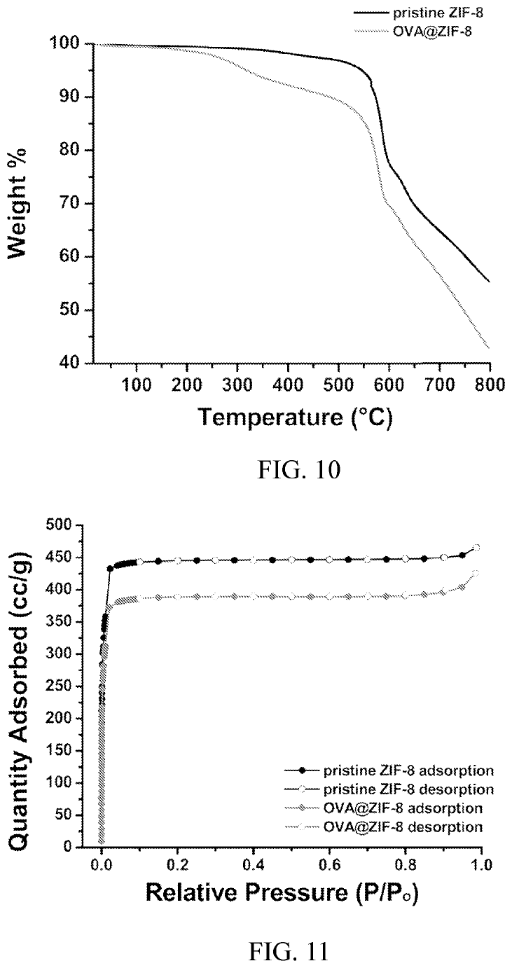

Thermogravimetric analysis (TGA, TA Instruments Q500 Thermal Analysis System) showed the thermal stability and degradation profile of OVA@ZIF-8 particles and suggested that OVA@ZIF-8 particles were thermally stable until approximately 250° C. at which the OVA@ZIF-8 particles decomposed due to decomposition of OVA (FIG. 10). These results suggested that OVA@ZIF-8 particles decomposed at lower temperatures than pristine ZIF-8, but the failure temperature was significantly higher than the expected temperatures during vaccine storage and distribution.

Vaccine loading efficiency of OVA@ZIF-8 particles (99.82%) was calculated as:

$$( ( Initial mass of vaccine - Remaining mass of vaccine in supernatants ) / Initial mass of vaccine ) * 100 ) .$$

And Vaccine loading capacity of OVA@ZIF-8 particles (8%) was determined as:

(Loading Efficiency*Initial Mass of Vaccine)/Mass of MOF Particles)*100).

These results agreed with weight reduction observed in thermogravimetric analysis of OVA@ZIF-8 at elevated temperatures due to the decomposition of OVA from ZIF-8.

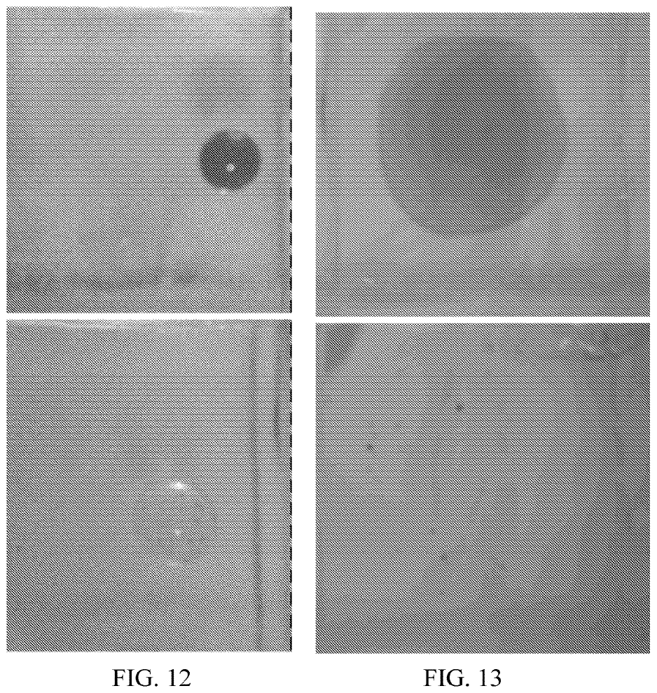

Nitrogen adsorption/desorption isotherms (Micromeritics 3Flex Gas Absorption Analyzer) indicated that OVA@ZIF-8 particles exhibited a Type-I isotherm like pristine ZIF-8 particles, and as expected, OVA@ZIF-8 particles were less porous than pristine ZIF-8 particles, with a Brunauer-Emmett-Teller (BET) surface area of approximately 1,850 m²/g, due to vaccine loading (FIG. 11).

### Example 3

### Synthesis of Microneedle-Array-Metal-Organic Framework-Vaccine Biocomposites

Microneedle array-metal-organic framework-vaccine biocomposites were prepared using a three-stage manufacturing strategy.

### Fabrication of Master Molds and Production Molds

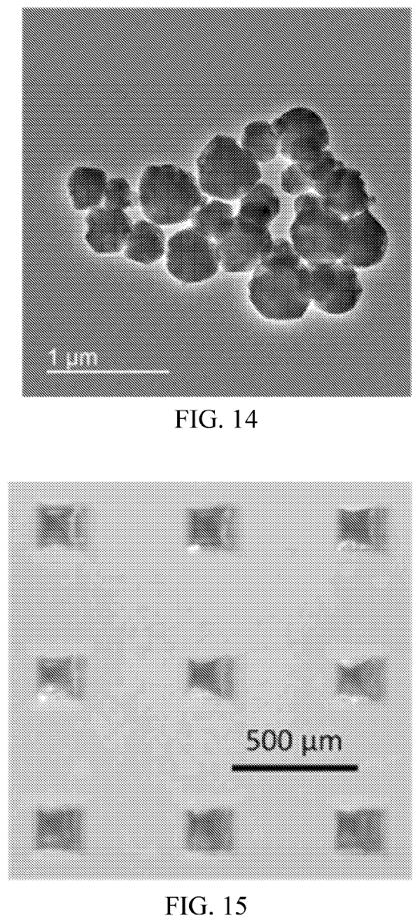

Master molds of microneedle array-metal-organic framework-vaccine biocomposites were prepared using microfabrication strategies, including 3D laser printing, photolithography, dry etching, or micromachining, to dictate microneedle and array geometries, as well as the dimensions of microneedles and their spatial distribution across the array. Production molds of microneedle array-metal-organic framework-vaccine biocomposites, with microneedle-shaped wells/cavities were prepared via replicating master molds using micromolding of different hydrophobic or hydrophilic materials to control the surface interactions between the production molds and metal-organic framework-vaccine biocomposites (FIGS. 12 and 13). The candidate materials for production molds include EcoFlex (hydrophobic), polydimethylsiloxane (PDMS, hydrophobic), plasma-treated PDMS (more hydrophilic than PDMS), a copolymer of PDMS and poly(ethylene glycol) (PEG) (more hydrophilic than PDMS), and Agarose (hydrophilic).

### Manufacturing of Microneedle Array-Metal-Organic Framework-Vaccine Biocomposites

Metal-organic framework-vaccine biocomposites were loaded into production molds of microneedle arrays in multiple ways, including:
1) Metal-organic framework-vaccine biocomposites were resuspended in water-soluble/hydrophilic biomaterials/surfactants (carboxymethyl cellulose (CMC), CMC and trehalose, CMC and sucrose, CMC and maltodextrin, CMC and lactose, hyaluronic acid, chitosan, alginate, gantrez, polyvinylpyrrolidone (PVP), polyvinyl alcohol (PVA), PVP and PVA) or nucleic acid adjuvants (Poly(I:C) or CpG) to coat them with hydrophilic compounds to overcome the hydrophobic/hydrophobic interaction and charge interactions with production molds of microneedle arrays;
2) More hydrophilic materials (plasma treated PDMS or PDMS-PEG) were used as the materials of the production molds;
3) Metal-organic framework-vaccine biocomposites were resuspended in lower density organic solvents (ethanol or acetonitrile or methanol or) as they are stable in organic solvents; and
4) The individual components of metal-organic framework-vaccine biocomposites were loaded into molds for in-situ synthesis of metal-organic framework-vaccine biocomposites.

Finally, the structural material of microneedle arrays (CMC, CMC and trehalose, CMC and sucrose, CMC and maltodextrin, CMC and lactose, hyaluronic acid, chitosan, alginate, gantrez, PVP, PVA, PVP and PVA, poly(lactic-co-glycolic acid) were loaded to form the rest of microneedles and the backing layer.

### Characterization of Microneedle-Array-Organic Framework-Vaccine Biocomposites

Optical stereomicroscopy (ZEISS Stemi 2000-C microscope with Olympus OM-D E-M5 II camera) and fluorescence microscopy (Keyence BZ-X700) analyses of production molds and dissolvable microneedle array-metal-organic framework-vaccine biocomposites demonstrated:
1) the effect of surface modification of the production molds on improved vaccine spreading;
2) successful loading of the production molds with metal-organic framework-vaccine biocomposites; and
3) high-quality of final dissolvable microneedle array-metal-organic framework-vaccine biocomposites.

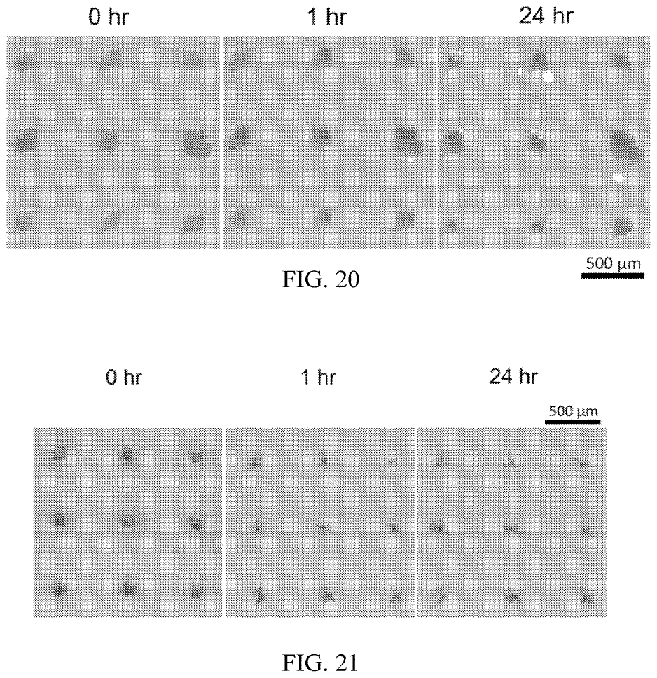

In vitro release testing in 4% agarose showed the stability of dissolvable microneedle array-metal-organic framework-vaccine biocomposites in aqueous environments compared to vaccine-loaded dissolvable microneedle arrays (FIGS. 20 and 21).

### Example 4

### Skin-Targeted Delivery Performance of Microneedle Array-Metal-Organic Framework Vaccine Biocomposites was Evaluated in Mouse Skin In Vivo and in Human Skin Samples Ex Vivo

Dissolvable microneedle array-zeolitic imidazolate framework-8-Alexa Fluor 647 labeled ovalbumin biocomposites (OVA AF647@ZIF-8@MNA) were prepared from a mixture of two water-soluble materials, carboxymethyl cellulose (CMC) and trehalose, which are designated as generally recognized as safe ‘GRAS’ by regulatory agencies, using the three-stage fabrication strategy described herein and applied to mouse skin in vivo and to freshly excised human skin explants ex vivo.

### In Vivo Testing in Mouse Skin

Optical stereomicroscopy (ZEISS Stemi 2000-C microscope with Olympus OM-D E-M5 II camera) analysis of OVA AF647@ZIF-8@MNA biocomposites before and after application to murine skin in vivo demonstrated that OVA AF647@ZIF-8@MNAs integrate a design and biomaterial with ideal physicochemical properties (sufficient mechanical strength for failure-free skin penetration and efficient solubility in aqueous environment) for effective cutaneous penetration to mouse skin in vivo and subsequent dissolution in the skin microenvironment.

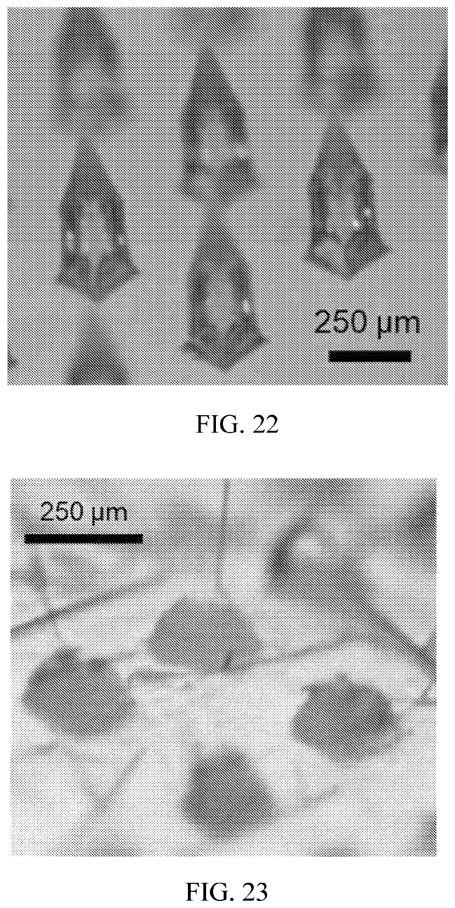

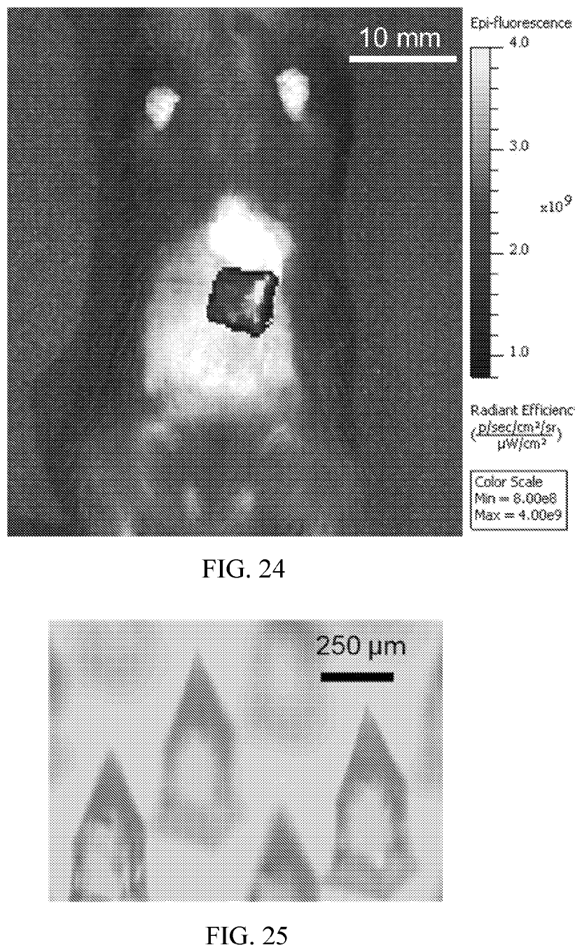

In vivo live animal fluorescent imaging (IVIS Lumina XR) analysis of OVA AF647@ZIF-8@MNA biocomposite-treated mice showed that OVA AF647@ZIF-8@MNA biocomposites were capable of depositing OVA AF647@ZIF-8 to mouse skin (FIGS. 22-24).

### Ex Vivo Testing in Freshly Excised Living Human Skin Explants

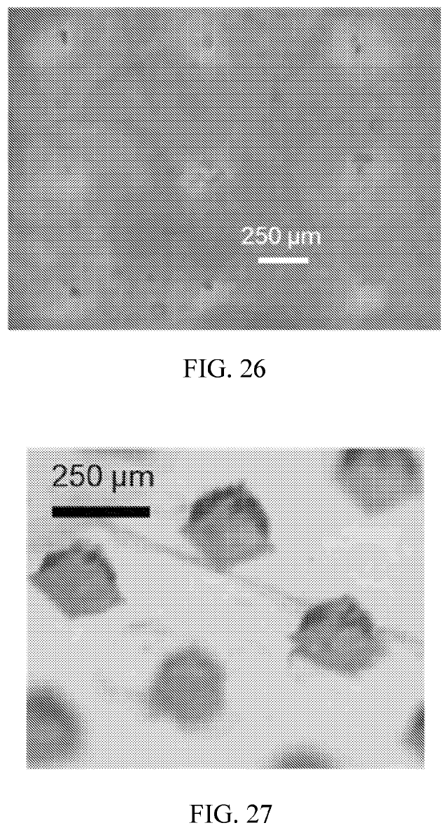

Optical stereomicroscopy (ZEISS Stemi 2000-C microscope with Olympus OM-D E-M5 II camera) analysis of OVA AF647@ZIF-8@CMC/Trehalose MNA biocomposites before (FIG. 25) and after (FIGS. 26 and 27) application to freshly excised human skin samples showed that OVA AF647@ZIF-8@CMC/Trehalose MNAs integrate a design and biomaterial with ideal physicochemical properties (sufficient mechanical strength for failure-free cutaneous penetration and efficient solubility in aqueous environment) for effective human skin penetration and subsequent dissolution.

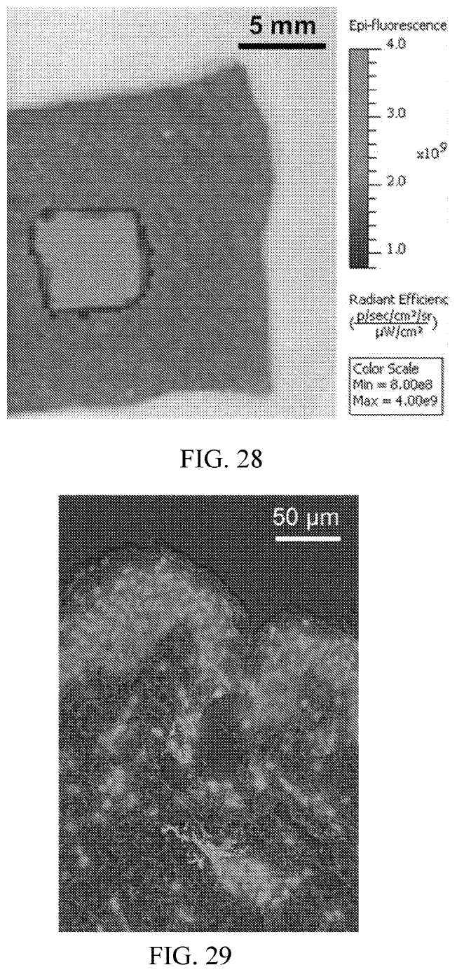

Optical stereomicroscopy (ZEISS Stemi 2000-C microscope with Olympus OM-D E-M5 II camera) and fluorescent imaging (IVIS Lumina XR) analyses of OVA AF647@ZIF-8@MNA biocomposite-treated human skin samples showed that OVA AF647@ZIF-8@ MNA biocomposites were capable of depositing OVA AF647@ZIF-8 to human skin. IVIS fluorescent imaging analysis also showed that MNAs more effectively achieve spatial distribution of OVA AF647@ZIF-8 compared with intradermal injection of OVA AF647@ZIF-8 with traditional hypodermic needles (FIG. 28).

Cryosectioning followed by fluorescent microscopy (Keyence BZ-X700) analysis of OVA AF647@ZIF-8@MNA biocomposite-treated human skin samples demonstrated that OVA AF647@ZIF-8@CMC/Trehalose MNA biocomposites pierced the human skin and delivered OVA AF647@ZIF-8 to the immunologically rich human skin microenvironments (FIG. 29).

These results suggest that dissolvable microneedle array-metal-organic framework-vaccine biocomposites afforded the precise targeting of metal-organic framework-vaccine biocomposites to immunocompetent microenvironments in mouse and human skin.

### Example 5

### In Vivo Evaluation of Immunogenicity and Safety Assessment of Dissolvable Microneedle Array-Metal-Organic Framework Vaccine Biocomposites

Biodissolvable microneedle array-zeolitic imidazolate framework-8-Ovalbumin biocomposites (OVA@ZIF-8@MNA) were prepared from a mixture of two water-soluble biomaterials, carboxymethylcellulose and trehalose, which are designated as generally recognized as safe “GRAS” by the regulatory agencies, using the three-stage manufacturing strategy as described herein. Dissolving microneedle arrays integrating Ovalbumin (OVA@MNA) were also prepared from the same mixture of carboxymethylcellulose and trehalose using the three-stage manufacturing strategy.

### Immunogenicity of Biodissolvable Microneedle Array-Metal-Organic Framework-Vaccine Biocomposites

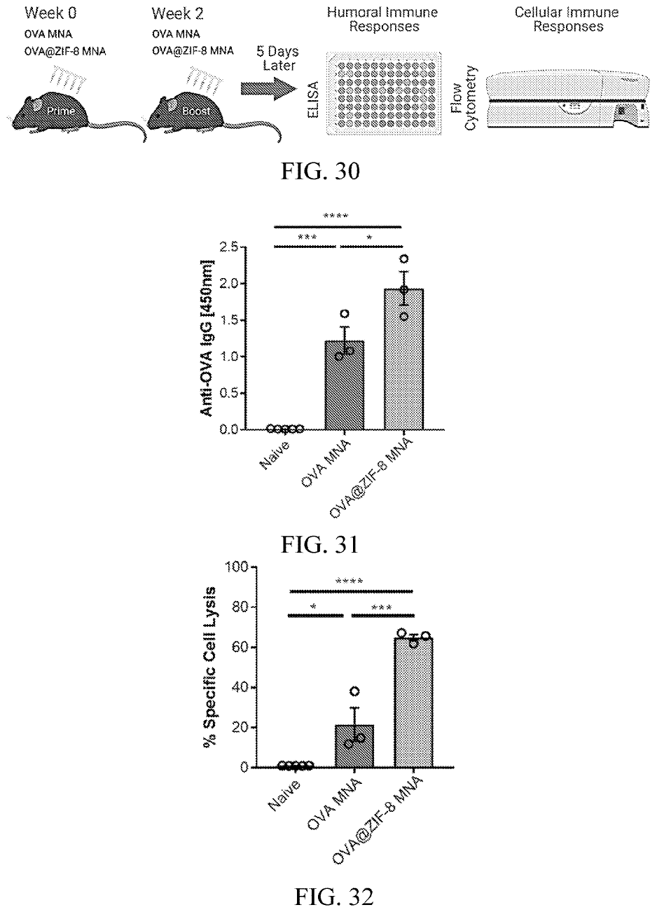

Humoral immune responses: C57BL/6 female mice (N=3 mice per group) received two doses of 1) OVA via intramuscular injection (OVA IM), 2) OVA@MNA, and 3) OVA@ZIF-8@MNA two weeks apart. Naïve mice served as unimmunized controls. 5 days after the booster dose, vaccine-induced antibody responses were evaluated by ELISA measurements of anti-OVA total IgG antibodies in mice sera (FIGS. 30-31).

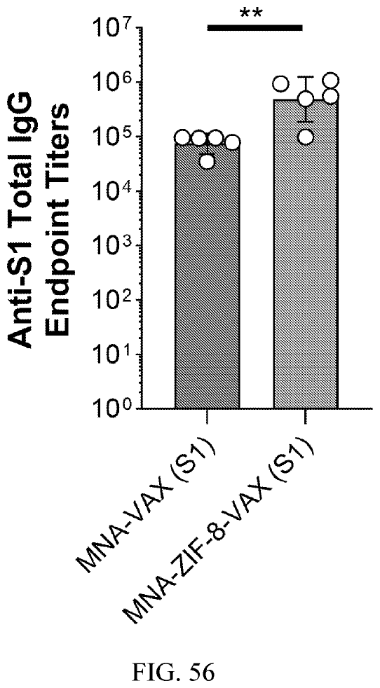

Humoral immune responses: C57BL/6 female mice (N=5 mice per group) received the primary dose of 1) Two MNA-VAX (S1) and 2) Two MNA-ZIF-8-VAX (S1). Naïve mice served as unimmunized controls. Two weeks after the primary dose, vaccine-induced antibody responses were evaluated by ELISA measurements of anti-SARS-COV-2-S1 total IgG antibodies in mice sera (FIG. 56).

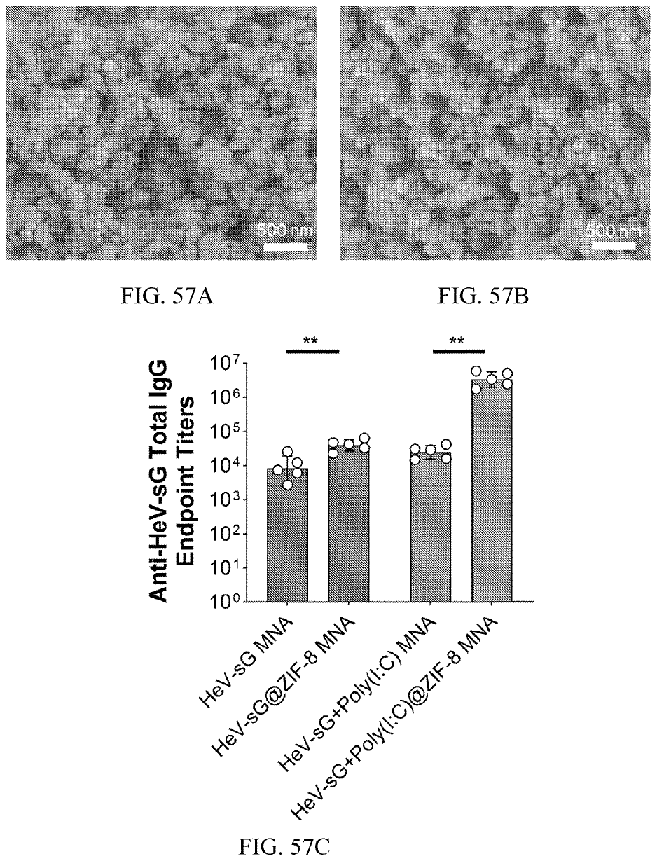

Humoral immune responses: C57BL/6 female mice (N=5 mice per group) received the primary dose of 1) HeV-sG MNA, 2) HeV-sG@ZIF-8 MNA, 3) HeV-sG+Poly(I:C) MNA, and 4) HeV-sG+Poly(I:C)@ZIF-8 MNA. Naïve mice served as unimmunized controls. Four weeks after the primary dose, vaccine-induced antibody responses were evaluated by ELISA measurements of anti-HeV-sG total IgG antibodies in mice sera (FIG. 57C).

Cellular immune responses: C57BL/6 female mice (N=3 mice per group) received two doses of 1) OVA@ZIF-8@MNA and 2) OVA@MNA two weeks apart. Naïve mice served as unimmunized controls. To determine activity of OVA-specific cytotoxic T lymphocytes (CTLs), equal numbers of unpulsed splenocytes (CFSE^{low} “control” cells) and OVA₂₅₇₋₂₆₄ peptide-pulsed splenocytes (CFSE^{high} “target” cells) were transferred to naïve and immunized mice (2×10⁷ total cells per mouse) 5 days after the booster dose. Spleens were isolated the next day and quantification of specific cell lysis, with 100% lysis corresponding to complete elimination of target cells, was performed. (FIG. 30 and FIG. 32).

Results from tests were compared by one-way ANOVA, followed by Tukey's post-hoc tests. Significant differences are indicated by * p<0.05, ** p<0.01, *** p<0.001, or **** p<0.0001.

### Safety of Dissolvable Microneedle Array-Metal-Organic Framework-Vaccine Biocomposites

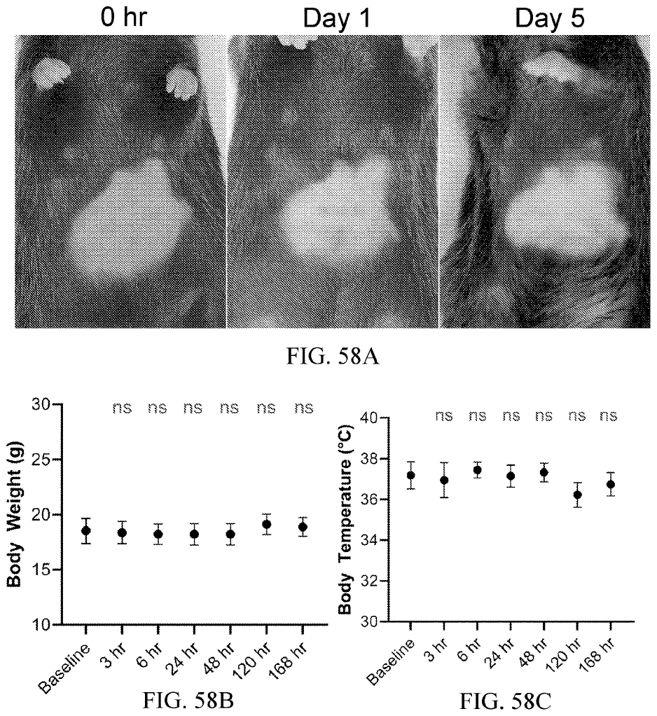

No abnormal animal behavior, no detectable weight loss, and no visible signs of overt skin and systemic reactogenicity were observed during these immunogenicity studies (FIGS. 58A-58C).

With the advantages of facile synthesis, chemical robustness, thermostability, and biocompatibility, dissolvable microneedle array-metal-organic framework-vaccine biocomposites pave the way for the development of next-generation vaccines with enhanced immunogenicity and avoiding cold chain storage and distribution.

### Example 6

### Reproducible and Facile Synthesis of Vaccine-Loaded Zeolitic Imidazolate Framework-8 (ZIF-8) was Achieved Using a Simple, Cost-Effective, and Scalable Protocol with Aqueous Solutions

A 3M solution of 2-methylimidazole was prepared by sonicating a mixture of 2-methylimidazole (246.3 mg, 3 mmol) in 1 mL nuclease-free water (H₂O). A 1M solution of zinc acetate dihydrate was prepared by sonicating a mixture of zinc acetate dihydrate (183.5 mg, 1 mmol) in 1 mL nuclease-free water. The vaccine solution was prepared by at the desired concentration. 3M 2-methylimidazole solution (853 μL, 2.56 mmol) and Ag solution (67 μl) were added to a 1.5 mL Eppendorf microcentrifuge tube in sequence and the tube was vortexed for 10 seconds. Then 1M zinc acetate dihydrate (80 μL, 0.08 mmol) was added, and the tube was vortexed again for 30 seconds. The solution reacted at room temperature or at lower temperature for 1 hour to yield a turbid solution. The suspension was centrifuged at 10,000 rpm for 2 minutes. The precipitate was washed with nuclease-free water (2 mL, 2×) and ethanol or methanol (2 mL, 2×) and dried under vacuum to obtain subunit vaccine@ZIF-8 crystals as a precipitate.

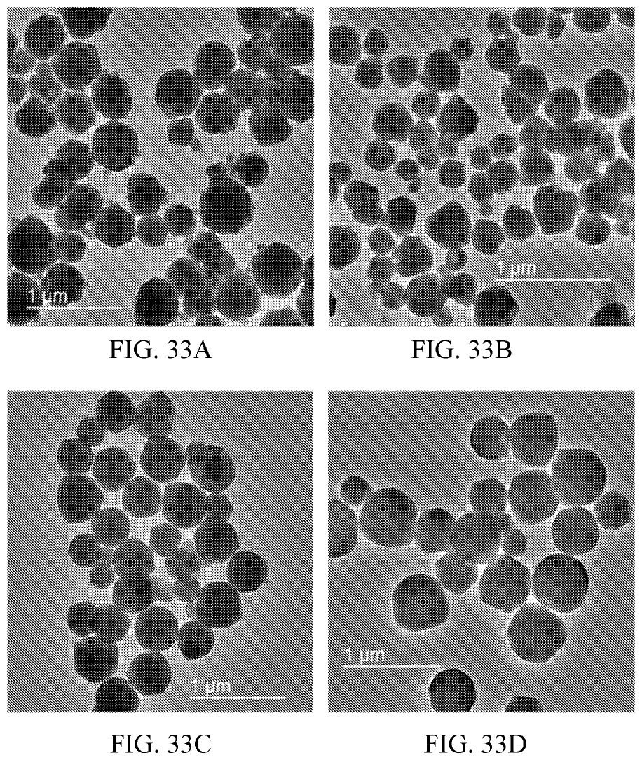

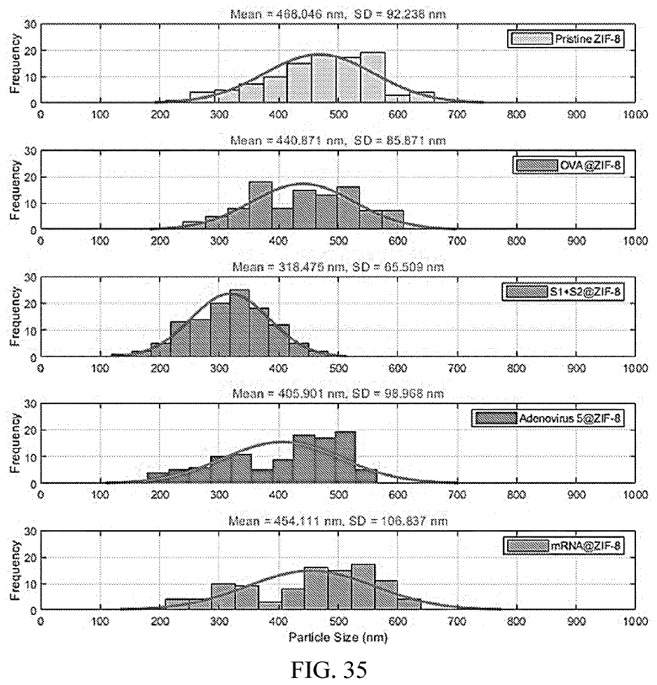

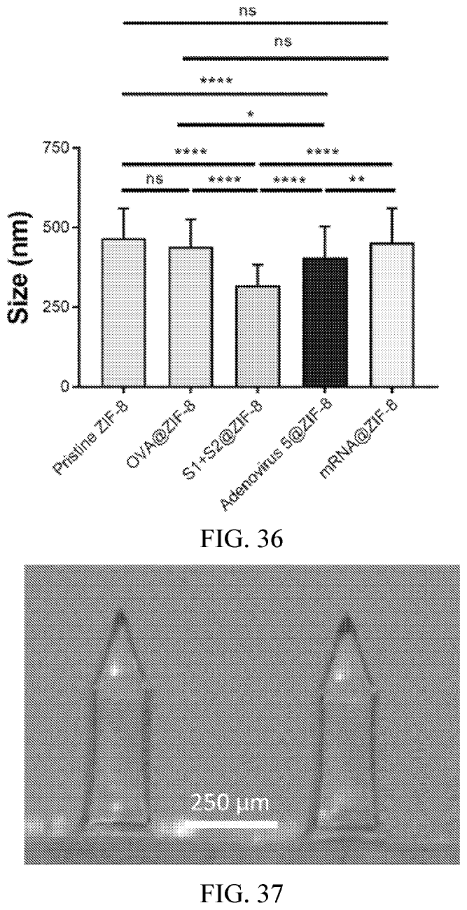

Transmission electron microscopy (TEM, An FEI Morgagni 268) analysis showed successful formation of vaccine-loaded ZIF-8 particles with an average size distribution of 441±86 nm (OVA@ZIF-8 particles), 318±66 nm (SARS-COV-2 S1+S2@ZIF-8 particles), 406±99 nm (Adenovirus5@ZIF-8 particles), 454±107 nm (mRNA@ZIF-8 particles). There were differences between the size of ZIF-8 particles prepared with different types of biomolecules (FIGS. 33A-33D and 35-36).

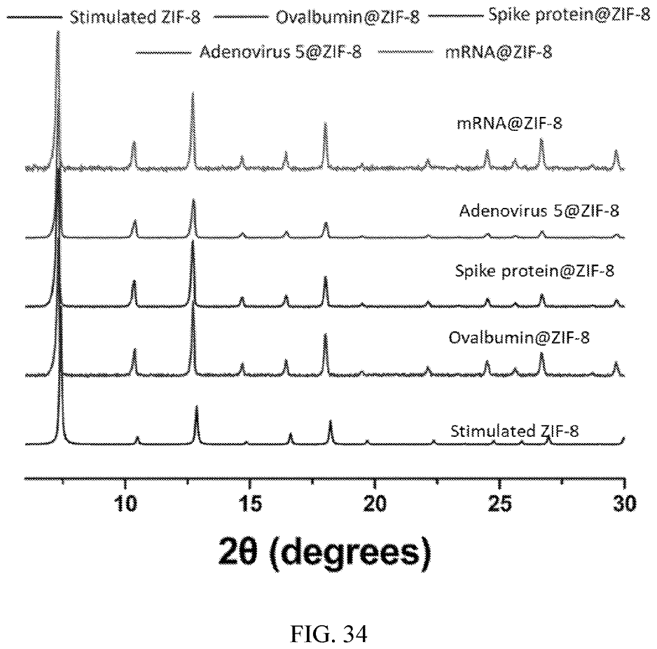

Powder X-ray diffraction (PXRD, A Bruker AXS D8 Discover X-Ray Powder Diffractometer) analysis showed that patterns of vaccine@ZIF-8 particles match the simulated results, indicating that Vaccube@ZIF-8 particles were highly crystalline and phase pure (FIG. 34). Vaccine loading efficiency of OVA@ZIF-8 particles (99.82%) and mRNA@ZIF-8 particles (97.91%) was calculated as:

$$( Initial mass of vaccine - Remaining mass of vaccine in supernatants ) / Initial mass of vaccine ) * 100 ) .$$

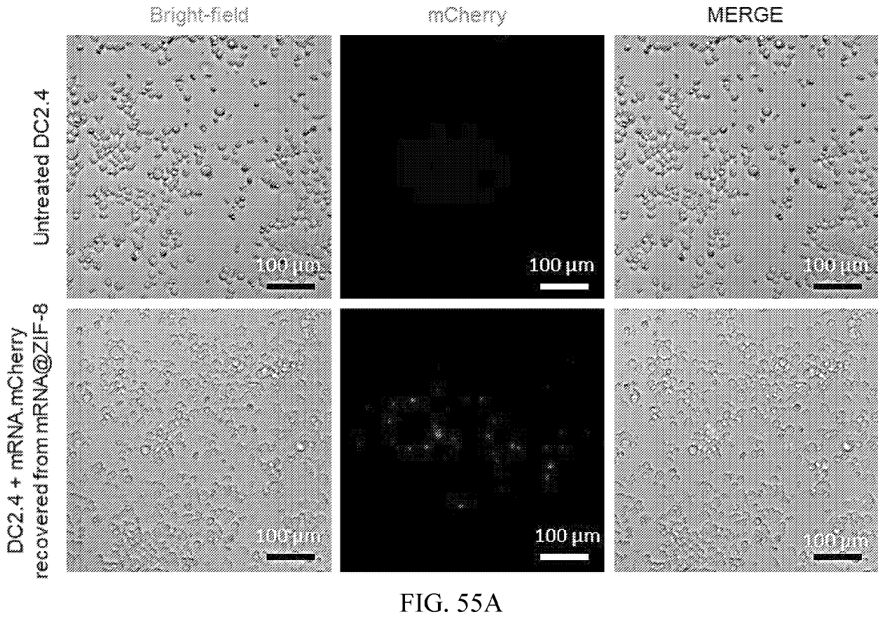

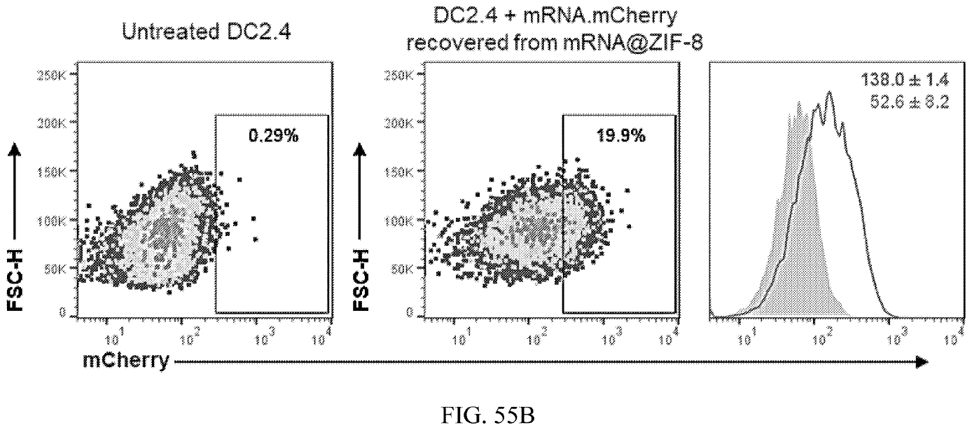

Fluorescence microscopy and flow cytometry analysis of DC 2.4 cells transfected with mRNA.mCherry (i.e., mRNA vector encoding the gene for mCherry) recovered from mRNA@ZIF-8 showed the expression of mCherry (FIGS. 55A-B), demonstrating the retention of the integrity of mRNA during the MOF encapsulation.

### Example 7

### Manufacturing and Characterization of Microneedle Array-Metal-Organic Framework-Vaccine Biocomposites with Various Antigen Constructs

### Synthesis of Dissolvable Microneedle-Array-Metal-Organic Framework-Vaccine Biocomposites

Microneedle array-metal-organic framework-vaccine biocomposites with various antigen constructs (Ovalbumin protein SARS-COV-2 subunit protein, Adenovirus 5 viral vector, and mRNA nucleic acid) were prepared using the multi-stage manufacturing strategy disclosed herein. Dissolvable MNAs with obelisk-shaped microneedles integrating ZIF-8 loaded with different biomolecules were manufactured from a biomaterial combination of two FDA-designated “Generally Recognized as Safe” (GRAS) biomaterials, Carboxymethylcellulose (CMC) and trebalose using the three-stage (master mold-production mold-dissolvable MNAs) microfabrication technique.

### Characterization of Microneedle-Array-Organic Framework-Vaccine Biocomposites

Optical stereomicroscopy (ZEISS Stemi 2000-C microscope with Olympus OM-D E-M5 II camera) and fluorescence microscopy (Keyence BZ-X700) analyses of dissolvable microneedle array-metal-organic framework-vaccine biocomposites with different antigen constructs demonstrated the high-quality of dissolvable microneedle array-metal-organic framework-vaccine biocomposites with different antigen constructs, including ovalbumin protein SARS-COV-2 subunit protein, Adenovirus 5 viral vector, and mRNA nucleic acid (FIGS. 37-42).

### Example 8

### Intracutaneous Delivery Performance of Dissolvable Microneedle Array-Metal-Organic Framework-SARS-COV-2 Vaccine Biocomposites

Skin-targeted delivery performance of microneedle array-metal-organic framework SARS-CoV-2 vaccine biocomposites was evaluated in mouse skin in vivo and in freshly excised human skin samples ex vivo.

Dissolvable microneedle array-zeolitic imidazolate framework-8-Alexa Fluor 647 labeled SARS-COV-2 S1 subunit vaccine biocomposites (S1 AF647@ZIF-8@MNA) were prepared from a mixture of two water-soluble materials, carboxymethylcellulose (CMC) and trehalose, which are designated as generally recognized as safe “GRAS” by the regulatory agencies, using the three-stage manufacturing strategy described above.

### In Vivo Testing in Mouse Skin

In vivo live animal fluorescent imaging (IVIS Lumina XR) analysis of S1@ZIF-8@MNA biocomposite-treated mouse showed that S1 AF647@ZIF-8@CMC/Trehalose MNA biocomposites are capable of depositing SARS-COV-2 S1 AF647@ZIF-8 to mouse skin. (FIG. 43).

### Ex Vivo Testing in Freshly Excised Living Human Skin Explants

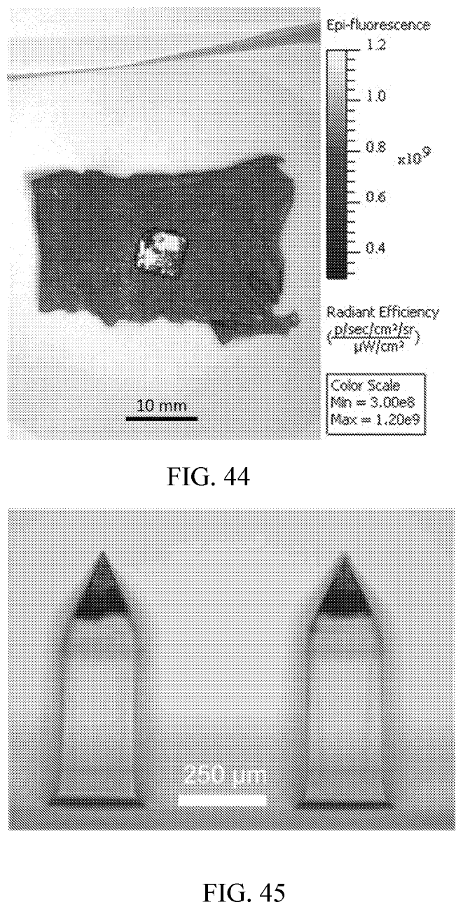

Fluorescent imaging (IVIS Lumina XR) analyses of S1 AF647@ZIF-8@MNA biocomposite-treated human skin samples showed that S1 AF647@ZIF-8@CMC/Trehalose MNA biocomposites were capable of depositing SARS-COV-2 S1 AF647@ZIF-8 to human skin. (FIG. 44).

Thus, these results showed that dissolvable microneedle array-metal-organic framework-vaccine biocomposites afforded the reliable and consistent skin-targeted delivery of metal-organic framework-SARS-COV-2 vaccine biocomposites to immunocompetent microenvironments in mouse and human skin.

### Example 9

### Manufacturing and Characterization of Rapidly Separable Dissolvable Microneedle Array-Metal-Organic Framework-Vaccine Biocomposites

### Synthesis of Rapidly Separable Microneedle-Array-Metal-Organic Framework-Vaccine Biocomposites

Separable microneedle array-metal-organic framework-vaccine biocomposites were prepared using the three-stage manufacturing strategy disclosed here.

### Fabrication and Production of Master Molds

Master molds and production molds of microneedles were prepared as described herein.

### Manufacturing of Separable Microneedle Array-Metal-Organic Framework-Vaccine Biocomposites

Separable metal-organic framework-vaccine biocomposites were manufactured using the approach described herein for dissolvable microneedle array-metal-organic framework-vaccine biocomposites with modifications in the last step. Biomineralization of zeolitic imidazolate framework-8-Alexa Fluor 647 labeled ovalbumin (OVA AF647@ZIF-8) was achieved as described herein and rhodamine-labeled negatively charged polyinosinic-polycytidylic acid (Poly(I:C) Rhodamine) was absorbed to OVA AF647@ZIF-8 via electrostatic interactions (OVA AF647@ZIF-8-Poly(I:C)Rhodamine). MNA-MOF-vaccine biocomposites were then prepared with water-soluble biomaterials that are designated as generally recognized as safe ‘GRAS’ by the regulatory agencies (e.g., carboxymethylcellulose (CMC), CMC and trehalose, CMC and sucrose, CMC and lactose, CMC and sucrose, hyaluronic acid (HA), or PVP or PVP and PVA) and integrated with water insoluble and organic solvent soluble (e.g., ethyl lactate) thermoplastics (e.g., poly(lactic-co-glycolic acid), polymethyl methacrylate, or polylactic acid) on PMMA stem) as part of the scalable manufacturing process.

### Characterization of Microneedle Array-Metal-Organic Framework-Vaccine Biocomposites

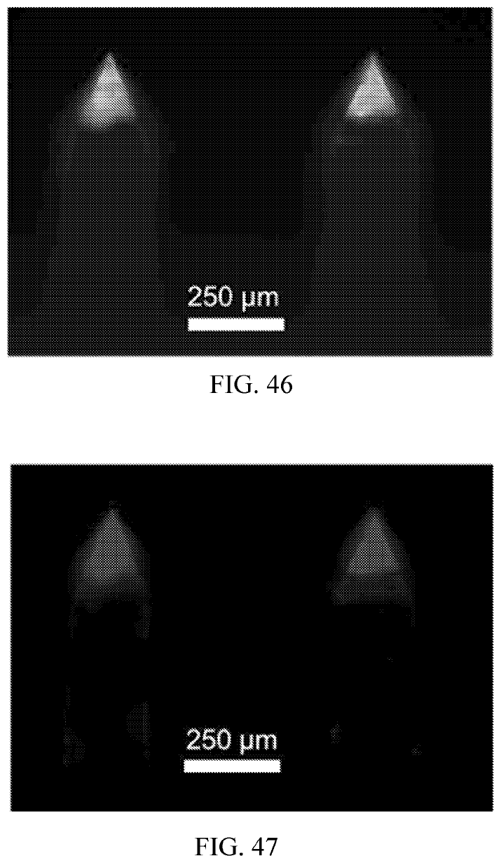

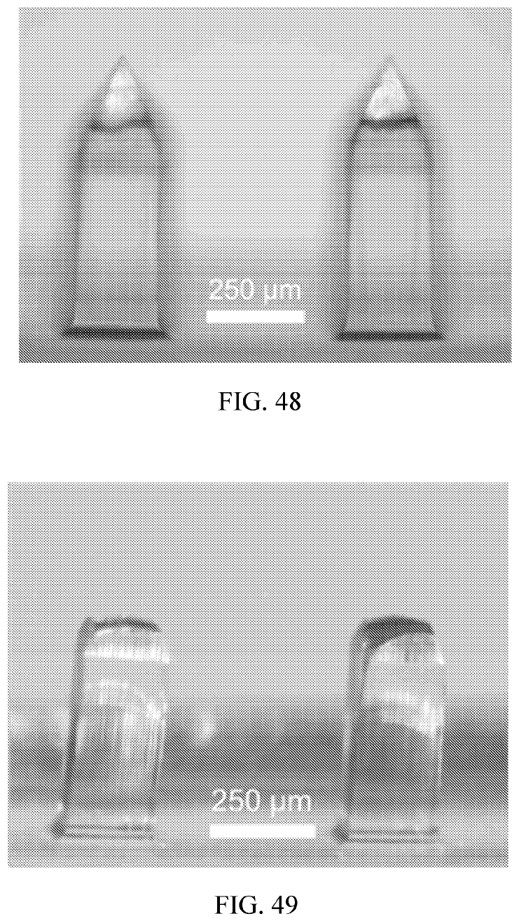

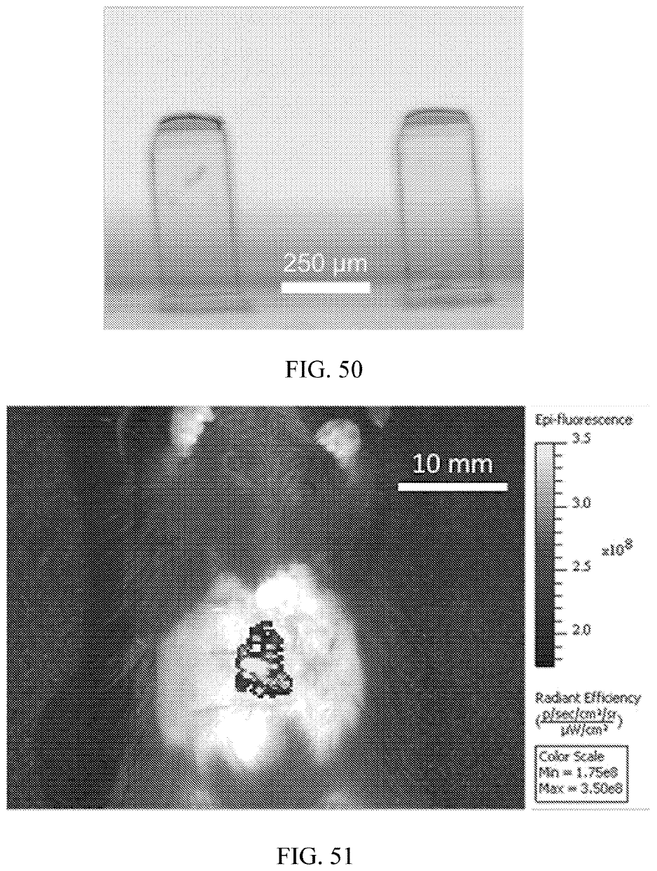

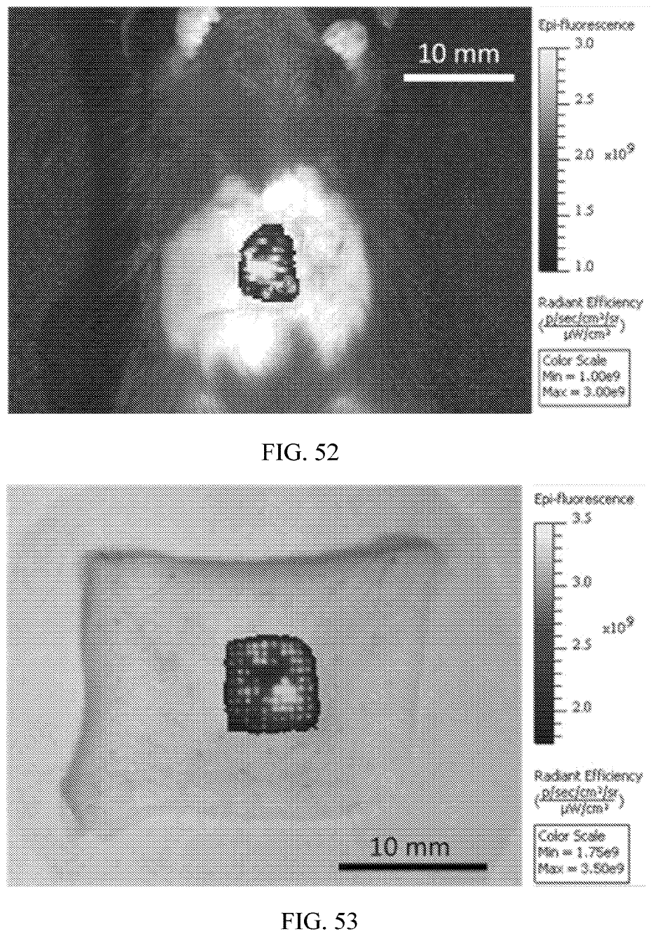

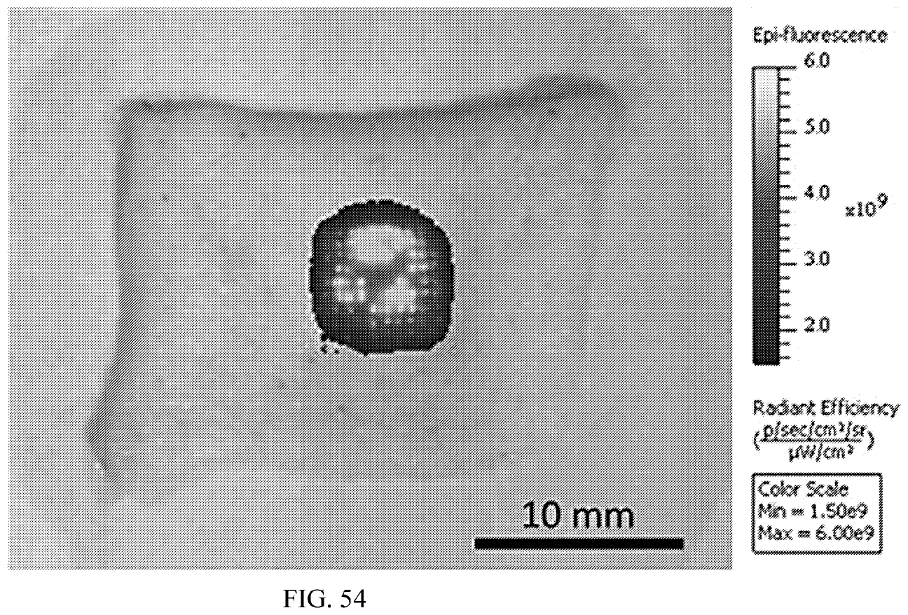

Optical stereomicroscopy (ZEISS Stemi 2000-C microscope with Olympus OM-D E-M5 II camera) and fluorescence microscopy (Keyence BZ-X700) analysis of separable microneedle array-metal-organic framework-vaccine biocomposites showed successful formation and application of separable microneedle array-metal-organic framework-vaccine biocomposites (FIGS. 45-50).

### Example 10

### Cutaneous Vaccine Delivery with Separable Microneedle Array-Metal-Organic Framework-Vaccine Biocomposites

Skin-Targeted Vaccine Delivery with Separable Microneedle Array-Metal-Organic Framework Vaccine Biocomposites was Evaluated in Mouse Skin In Vivo and in Freshly Excised Human Skin Samples Ex Vivo

Separable microneedle array-zeolitic imidazolate framework-vaccine biocomposites were prepared using the multi-stage manufacturing strategy described herein.

### In Vivo Testing in Mouse Skin

In vivo live animal fluorescent imaging (IVIS Lumina XR) analysis of separable MNA-MOF-vaccine biocomposites (OVA AF647@ZIF-8-Poly(I:C)Rhodamine@CMC/Trehalose biocomposites on PMMA or OVA AF647 @ZIF-8-Poly(I:C)Rhodamine@PVP biocomposites on PMMA) treated mice showed that these separable MNA-MOF-vaccine biocomposites were capable of depositing OVA AF647@ZIF-8-Poly(I:C)Rhodamine@water-soluble material biocomposites to murine skin in vivo.

### Ex Vivo Testing in Freshly Excised Living Human Skin Explants

Fluorescent imaging (IVIS Lumina XR) analysis of separable MNA-MOF-vaccine biocomposites (OVA AF647@ZIF-8-Poly(I:C)Rhodamine@CMC/Trehalose biocomposites on PMMA or OVA AF647 @ZIF-8-Poly(I:C)Rhodamine@PVP biocomposites on PMMA) treated human skin samples showed that these novel separable MNA-MOF-vaccine biocomposites were capable of rapidly depositing OVA AF647@ZIF-8-Poly(I:C)Rhodamine@water-soluble material biocomposites to human skin ex vivo.

Together, these results demonstrate that these sophisticated separable MNA-MOF-vaccine biocomposites afford the precise and rapid targeting of MOF-multi-component vaccine-biocomposites to both human and murine skin microenvironments with improved skin delivery characteristics.

In view of the many possible aspects to which the principles of the disclosed technology may be applied, it should be recognized that the illustrated aspects are only preferred examples of the technology and should not be taken as limiting the scope of the technology. Rather, the scope of the technology is defined by the following claims. We therefore claim as our disclosure all that comes within the scope and spirit of these claims.

1. A microneedle array comprising:
a base portion;
a plurality of biocompatible dissolvable microneedles extending from the base portion, wherein the microneedles comprise metal-organic framework (MOF) particles comprising a vaccine composition.

2. The microneedle array of claim 1, wherein the MOF is a zinc-based MOF, a copper-based MOF, or an iron-based MOF.

3. The microneedle array of claim 2, wherein the zinc-based MOF is ZIF-8, ZIF-10, ZIF-90; the copper-based MOF is HKUST-1; or the iron-based MOF is MIL-53, MIL-88.

4. The microneedle array of claim 1, wherein the MOF is surface functionalized with a surface coating comprising CMC, CMC/Trehalose, PEG, PVA, PVP, or hyaluronic acid, silk, or a combination thereof.

5. The microneedle array of claim 1, wherein the MOF particles have an average size distribution of from 10 nm to 50,000 nm.

6. The microneedle array of claim 1, wherein the base portion lacks the MOF particles comprising the vaccine composition.

7. The microneedle array of claim 1, wherein both the microneedles and the base portion integrate the MOF particles comprising the vaccine composition.

8. The microneedle array of claim 1, wherein the MOF particles comprising the vaccine composition are integrated into the tapered portion of the microneedles.

9. The microneedle array of claim 1, wherein the vaccine composition comprises an antigen selected from protein, mRNA, self-replicating mRNA, nucleic acid antigen, recombinant viral vectored antigen, nucleic acid origami antigen, extracellular vesicle antigen, inactivated virus antigen, liposome encapsulated antigen, extracellular vesicle encapsulated antigen, cell lysate antigen, bacterial antigen, or a combination thereof.

10. The microneedle array of claim 1, wherein the vaccine composition comprises an adjuvant.

11. The microneedle array of claim 1, wherein the microneedle array comprises:
a first plurality of MOF particles comprising a first vaccine composition; and
a second plurality of MOF particles comprising a second vaccine composition.

12. The microneedle array of claim 11, wherein the first vaccine composition comprises a first antigen, and the second vaccine composition comprises a second antigen.

13. The microneedle array of claim 11, wherein the first vaccine composition comprises an antigen, and the second vaccine composition comprises an adjuvant.

14. The microneedle array of claim 1, wherein each microneedle further comprises a dissoluble biocompatible material.

15. The microneedle array of claim 14, wherein the dissoluble biocompatible material is CMC, CMC and trehalose, CMC and sucrose, CMC and maltodextrin, CMC and lactose, hyaluronic acid, chitosan, alginate, gantrez, PVP, PVA, PVP and PVA, gelatin, poly lactic acid, pullulan, silk, polyphosphazene, poly-γ-glutamate, or poly(lactic-co-glycolic acid).

16. A method of fabricating a microneedle array according to claim 1, the method comprising:
forming a solution comprising a dissoluble biocompatible material and a plurality of metal-organic framework (MOF) particles comprising a vaccine composition; and
applying the solution to a microneedle array mold.

17. A method of fabricating a microneedle array according to claim 1, the method comprising adding a solution comprising a dissoluble biocompatible material to a microneedle array mold that contains a plurality of metal-organic framework particles comprising a vaccine composition, to form the microneedle array.

18. The method of claim 17, further comprising loading the plurality of metal-organic framework particles comprising the vaccine composition into the mold before adding the solution comprising the dissoluble biocompatible material.

19. The method of claim 17, further comprising loading into the mold precursors of the metal-organic framework and the vaccine composition to form the plurality of MOF particles comprising the vaccine composition in the mold before adding the solution comprising the dissoluble biocompatible material.

20. A method of vaccinating a subject, comprising applying the microneedle array of claim 1 to an area of the subject to deliver the vaccine composition to the subject.
